SEQUENCE LISTING (7 sequences)

SEQ ID NO: 1 - DNA, 21 bp, Mus sp.
aagtgtacaagtccgcgtcct

SEQ ID NO: 2 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 3 - DNA, 4247 bp, Homo sapiens
gttccccaactgctgttttattgtgctattcatgcctagacatcacatagctagaaaggcccatcagacccctcaggccactgctgttcctgtcacacattcctgcaaaggaccatgttgctaacttgaaaaaaattactattaattacacttgcagttgttgcttagtaacatttatgattttgtgtttctcgtgacagcatgagcagagatcattaaaaattaaacttacaaagctgctaaagtgggaagaaggagaacttgaagccacaatttttgcacttgcttagaagccatctaatctcaggtttatatgctagatcttgggggaaacactgcatgtctctggtttatattaaaccacatacagcacactactgacactgatttgtgtctggtgcagctggagtttatcaccaagacataaaaaaaccttgaccctgcagaatggcctggaattacaatcagatgggccacatggcatcccggtgaaagaaagccctaaccagttttctgtcttgtttctgctttctccctacagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaaaaaaaaaaaaaaaa

SEQ ID NO: 4 - DNA, 594 bp, Unknown
  Description of Unknown Woodchuck post- transcriptional regulatory element polynucleotide
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatc

SEQ ID NO: 5 - DNA, 19 bp, Unknown
  Description of Unknown Woodchuck post- transcriptional regulatory element oligonucleotide
ctatgtggacgctgcttta

SEQ ID NO: 6 - DNA, 19 bp, Unknown
  Description of Unknown Woodchuck post- transcriptional regulatory element oligonucleotide
tcctggtttgtctctttat

SEQ ID NO: 7 - DNA, 4755 bp, Homo sapiens
cacacacacacacacacacagagagaacatctctagtaaaaagaaaagttgagctttcttagctagatgtgtgtattagccagaaaaagccaaggagtgaagggttttagagaactggaggagataaagtggagtctgcatatgggaggcatttgaaatggacttaaatgtctttttaatgctgactttttcagttttctccttaccagacacattgttttcatgacattagccccaggcatagacacatcattaaaatgaacatgtcaaaaaatgatttctgtttagaaataagcaaaacattttcagttgtgaccacccaggtgtagaataaagaacagtggaattgggagccctgagttctaacataaactttcttcatgacataaggcaagtcttctatggcctttggtttccttacctgtaaaacaggatggctcaatgaaattatctttcttctttgctataatagagtatctctgtgggaagaggaaaaaaaaagtcaatttaaaggctccttatagttccccaactgctgttttattgtgctattcatgcctagacatcacatagctagaaaggcccatcagacccctcaggccactgctgttcctgtcacacattcctgcaaaggaccatgttgctaacttgaaaaaaattactattaattacacttgcagttgttgcttagtaacatttatgattttgtgtttctcgtgacagcatgagcagagatcattaaaaattaaacttacaaagctgctaaagtgggaagaaggagaacttgaagccacaatttttgcacttgcttagaagccatctaatctcaggttatatgctagatcttgggggcaaacactgcatgtctctggtttatattaaaccacatacagcacactactgacactgatttgtgtctggtgcagctggagtttatcaccaagacataaaaaaaccttgaccctgcagaatggcctggaattacaatcagatgggccacatggcatcccggtgaaagaaagccctaaccagttttctgtcttgtttctgctttctccctacagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaa

(19) United States
(12) Patent Application Publication
(10) Pub. No.: US 2011/0274718 A1
(43) Pub. Date: Nov. 10, 2011

(54) System for Modulating Expression of Hypothalmic Brain-Derived Neurotrophic Factor (BDNF)

(75) Inventors: Matthew J. During, Columbus, OH (US); Lei Cao, Columbus, OH (US)

(73) Assignee: THE OHIO STATE UNIVERSITY, Columbus, OH (US)

(21) Appl. No.: 13/126,026

(22) PCT Filed: Oct. 29, 2009
(86) PCT No.: PCT/US09/62514

§ 371 Date: Jul. 18, 2011

Related U.S. Application Data
(60) Provisional application No. 61/109,454, filed on Oct. 29, 2008.

(51) Int. Cl. A61K 39/00; A61K 31/7088; C12N 15/63; A61K 38/18; A61P 3/00; A61P 35/00; A61P 3/04; A61P 3/10; A61P 37/04; A61P 25/00; A61K 48/00; C07H 21/02

(52) U.S. Cl. 424/198.1; 514/44 R; 514/44 A; 435/320.1; 514/8.3; 536/24.5

(57) ABSTRACT

Methods for treating tumor associated diseases by administering a nucleic acid sequence encoding brain derived neurotrophic factor (BDNF) where the expression reduces the symptoms of the disease and compositions for mediation of enrichment-induced tumor resistance having brain derived neurotrophic factor (BDNF) are disclosed.

### CROSS-REFERENCE TO RELATED APPLICATIONS

This application claims the benefit of U.S. Provisional Application Ser. No. 61/109,454 filed Oct. 29, 2008, the entire disclosure of which is expressly incorporated herein by reference.

### STATEMENT REGARDING FEDERALLY SPONSORED RESEARCH

This invention was not made with any Government support and the Government has no rights in this invention.

### REFERENCE TO SEQUENCE LISTING

The instant application contains a sequence listing which has been submitted via EFS-web and is hereby incorporated by reference in its entirety. The ASCII copy, created on Oct. 26, 2009, is named 604_{—}50462_SEQ_LIST_OSURF_{—}08097.txt, and is 15,990 bytes in size.

### TECHNICAL FIELD AND INDUSTRIAL APPLICABILITY OF THE INVENTION

Described herein are methods for treating tumor associated diseases by administering a nucleic acid sequence encoding brain derived neurotrophic factor (BDNF). The expression reduces the symptoms of the disease. Also described herein are compositions for mediation of enrichment-induced tumor resistance having brain derived neurotrophic factor (BDNF).

### BACKGROUND OF THE INVENTION

The growth of most cancers is dependent, in part, on their microenvironment, that is, the balance between factors which act to facilitate growth, induce angiogenesis and cell survival, and those factors which act to inhibit cell proliferation and lead to apoptosis. This local microenvironment is influenced by systemic factors, and the cancer itself induces both local and distant changes through paracrine signaling and interactions with the immune and nervous systems. The effect of the macroenvironment on systemic cancer, specifically an individual's interaction with its physical living and social environment, is much less well defined.

There is significant interest in neuroscience in the interaction between genes and the environment, and specifically, how living in complex housing with increased physical and social activity influences brain structure and function. What is remarkable is just how robust and powerful the physical and social environment can have on brain function.

### SUMMARY OF THE INVENTION

In a first broad aspect, there are provided herein compositions useful for the mediation of enrichment-induced tumor resistance that includes an effective amount of brain derived neurotrophic factor (BDNF). The compositions are useful as a mediator linking the environment to metabolic and immune functions and tumor growth.

In another broad aspect, there is provided herein a method for treating a subject with a tumor-associated disease, comprising: administering an expression vector to a target cell in the subject. The expression vector comprises a nucleic acid sequence encoding brain derived neurotrophic factor (BDNF), or a derivative or functional fragment thereof. The administering results in expression of BDNF, or a derivative or functional fragment thereof, in the target cell. The expression reduces the symptoms of the neurological disease, thereby treating the subject with the disease.

In another broad aspect, there is provided herein a vector expressing microRNA targeting against mouse BDNF (miR-Bdnf) [SEQ ID NO:1].

In certain embodiments, the expression vector can be either a viral or a non-viral expression vector. In certain embodiments, the viral expression vector can be an adeno-associated virus (AAV) vector, a lentivirus vector, an adenovirus vector, or a herpes simplex virus (HSV) vector. In certain embodiments, the nucleic acid sequence encoding BDNF is a nucleic acid sequence encoding an amino acid sequence comprising BDNF brain-derived neurotrophic factor NM_{—}170735, Accession Numbers AB038670 Chromosome 11p14.1, or a derivative or a functional fragment thereof. In certain embodiments, the nucleic acid sequence encoding BDNF is a nucleic acid sequence encoding an amino acid sequence comprising [SEQ ID NO:2] or an amino acid sequence at least 80%-90% homologous to [SEQ ID NO:2]. The nucleotide sequence of NCBI LOCUS—NM_{—}170735, 4755 bp, mRNA, linear, PRI 15-FEB-2009; DEFINITION Homo sapiens brain-derived neurotrophic factor (BDNF), transcript, variant 1, mRNA; ACCESSION NM_{—}170735, VERSION NM_{—}170735.5 GI:219842286, is disclosed herein as [SEQ ID NO:7].

In certain embodiments, the disease is cancer, such as, but not limited to, melanoma, colon cancer.

In certain embodiments, the method includes administering by stereotaxic microinjection. In certain embodiments, the administering is by stereotaxic microinjection to a medial temporal lobe or temporal cortex of the central nervous system. In certain embodiments, the administering to the medial temporal lobe is localized to the hippocampus and/or amygdala.

In another broad aspect, there is provided herein a method for delivering a nucleic acid sequence to a mammalian target cell, wherein the nucleic acid sequence is expressible in the target cell for an extended period of time.

In certain embodiments, the method includes administering a BDNF-associated vector to the target cell, wherein the BDNF-associated vector transduces the target cell. Also, in certain embodiments, the BDNF-associated vector is free of both wildtype and helper virus.

In certain embodiments, the method includes administering a composition to at least one target cell, where the composition comprises a BDNF-associated vector capable of transducing the target cell.

In another broad aspect, there is provided herein a method for treating a subject with a cancer-related disease, comprising administering an BDNF-associated vector to a target cell in the subject, wherein the administering results in expression of BDNF, or a derivative or functional fragment thereof, in the target cell and the expression reduces the symptoms of the disease, thereby treating the subject with the disease.

In another broad aspect, there is provided herein a method of altering the expression of brain derived neurotrophic factor (BDNF) in of a subject comprising: identifying a target site in the subject that requires modification; delivering a vector comprising a nucleotide sequence encoding BDNF to the target site; and expressing BDNF in the target site.

In certain embodiments, the target site is a region of the brain selected from the group consisting of basal ganglia, subthalmic nucleus (STN), pedunculopontine nucleus (PPN), substantia nigra (SN), thalmus, hippocampus, cortex, and combinations thereof.

In another broad aspect, there is provided herein a method of altering expression of brain derived neutrophic factor (BDNF) in a of a subject having a disorder which causes morphological and/or functional abnormality of a cell or population of ells comprising: identifying a target site that requires modification; delivering a vector comprising a nucleotide sequence encoding BDNF to the target site; and expressing BDNF in the target site.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIGS. 1a-1e: Environmental enrichment enhances tumor resistance in mice:

FIG. 1a: Photograph of an enrichment cage. Mice were housed in the enrichment cage or control cages for 6 w before tumor inoculation.

FIG. 1b: Photographs showing representative B16 melanoma dissected d 17 after inoculation.

FIG. 1c: Graph showing that representative B16 melanoma dissected d 19 after inoculation, showing that enrichment decreased tumor volume d 19 after inoculation (n=20 in each group, * P<0.05).

FIG. 1d: Graph showing that enrichment further reduced tumor weight d 17 after inoculation (n=18 in each group, * P<0.05).

FIG. 1e: Graph showing that enrichment induced complete tumor resistance in a few mice. All control mice showed visible tumors.

FIGS. 2a-2d: Enrichment affects biomarkers in serum, B16 melanoma cell proliferation in vitro and signaling pathways and gene expression in the tumors:

FIG. 2a: Graph biomarkers in serum where sera were collected before tumor inoculation in the 6 week enrichment paradigms (n=20 in each group, * P<0.05).

FIG. 2b: Graph showing relative growth where B16 cells grew more slowly when cultured with serum from enrichment mice compared to control mice in both 3 w and 6 w enrichment paradigms (n=9-10 in each group, * P<0.05).

FIG. 2c: Graph showing relative concentration showing hospho-Akt1 (S473), ERK1 (T202/Y204)/ERK2 (T185/Y187), Phospho-p38α (T180/Y182), active HIF-1α activity and VEGF concentration were significantly reduced in tumors from enrichment mice compared to control mice (n=7 in each group, * P<0.05).

FIG. 2d: Graph showing relative mRNA expression, showing gene expression of melanoma associated transcription factor and antigens (n=8 in each group, * P<0.05, +P=0.07).

FIGS. 3a-3e: Enrichment enhances immunocompetence:

FIG. 3a: Graph showing stimulation index, showing the proliferative response of splenic lymphocytes to the T-cell mitogen Concanavalin A was increased in enrichment mice before and after tumor inoculation (n=5 in each group at each time point, * P<0.05, +P=0.054).

FIG. 3b: Graph showing spleen weights, showing that the spleens of enrichment mice were enlarged more than control mice d13 after tumor cells were inoculated (n=5 in each group, * P=0.05).

FIG. 3c: Photograph showing spleens used in graph of FIG. 3b.

FIG. 3d: Graph showing the percent of toxicity, showing that NK cytotoxicity was higher in enrichment mice (n=5 in each group, P<0.05) before tumor inoculation.

FIG. 3e: Graph showing the percent of toxicity, showing that CD8 T cell cytotoxicity was higher than control mice (bar represents a pool of 4 mice in each group, P<0.05).

FIG. 4a-4c: Graphs showing enrichment induces gene expression changes in arcuate nucleus of hypothalamus. Mice were housed in enrichment and control housing for 2 weeks (FIG. 4a), 4 weeks (FIG. 4b) and 9 weeks (FIG. 4c) (n=5 per group). P values of significance or strong trend were shown above the bars.

FIGS. 5a-5d: Hypothalamic gene delivery of BDNF mimics enrichment associated metabolic changes and melanoma resistance:

FIG. 5a: Graph showing biomarkers in serum, showing hypothalamic overexpression of BDNF leads to the similar serum biomarker changes as enrichment (n=10 in BDNF mice, n=16 in GFP mice, * P<0.05).

FIG. 5b: Graph showing relative growth (as a percent of GFP), showing B16 cells grew more slowly when cultured with serum from BDNF mice compared to GFP mice 4 weeks after AAV injection (n=5 in each group, * P<0.05).

FIG. 5c: Graph showing the relative tumor weight, showing that BDNF overexpression reduced tumor weight d 17 after inoculation (n=10 in BDNF mice, n=16 in GFP mice, * P<0.05).

FIG. 5d: Graph showing the stimulation index, showing that the proliferative response of splenic lymphocytes to the T-cell mitogen Concanavalin A was increased in BDNF mice (n=3 in each group, * P<0.05).

FIGS. 6a-6e: RNAi knockdown of BDNF expression in hypothalamus inhibits enrichment-induced tumor resistance:

FIG. 6a: Schematic illustration of experiment design.

FIG. 6b: Graph showing BDNF mRNA expression (percent of miR-scr control housing), using quantitative RT-PCR, showing that the miR-Bdnf vector significantly reduced hypothalamic BDNF mRNA level in mice housed in both control and enrichment condition (n=7-17 per group, * P<0.015 miR-Bdnf compared to miR-scr in both of control housing and enrichment, +P=0.061 miR-scr enrichment compared to miR-scr control housing).

FIG. 6c: Graph showing biomarkers in serum 4 weeks after AAV injection and 3 weeks enrichment (* P<0.05 enrichment housing compared to control housing).

FIG. 6d: Graph showing relative tumor weight, showing that miR-Bdnf blocks enrichment-induced tumor resistance. * P<0.05, mice treated with miR-scr and housed in enrichment compared to each of the other group. con, control housing, enr, enriched housing.

FIGS. 7a-7e: Effects of voluntary wheel running on metabolism, immune function and melanoma growth:

FIG. 7a: Graph showing biomarkers in serum, showing that running led to some changes in serum biomarkers which were distinctive to those observed in enrichment (n=16 in runners, n=13 in control mice, * P<0.05).

FIG. 7b: Graph showing relative mRNA expression, showing that running induced gene expression changes in the arcuate nucleus of hypothalamus. Mice were subjected to wheel running for 4 weeks (n=5 per group). P values of significance or strong trend were shown above the bars.

FIG. 7c: Graph showing relative tumor weight, showing that running did not reduce tumor growth. (n=11 in runner, n=10 in control mice, P>0.05).

FIG. 7d: Graph showing the stimulation index, showing that the proliferative response of splenic lymphocytes to the T-cell mitogen Concanavalin A was increased in runner (n=4 in each group, * P<0.05).

FIG. 7e: Graph showing the percent of cytotoxicity, showing that NK cytotoxicity was higher in runner (n=4 in each group, P<0.05) before tumor inoculation.

FIG. 8: Graph showing RNAi knockdown of BDNF protein in the hypothalamus. Graphs showing that BDNF protein levels were reduced by miR-Bdnf vector in both control and enrichment housing (n=7-17 per group * P<0.001 miR-Bdnf compared to miR-scr both in control housing and enrichment, +P=0.073 miR-scr enrichment compared to miR-scr control housing):

FIGS. 9a-9d: Graphs showing obesity related data:

FIG. 9a: Graph showing the AAV-BDNF treatment leads to marked weight loss of diet-induced obesity model (DIO) carrying colon cancer.

FIG. 9b: Graph showing AAV-BDNF treatment reduces adiposity particularly white fat. Epididymal white fat pad (EWAT) was reduced by 35.1±7.2% and retroperitoneal white fat pad (RWAT) was reduced by 52.2±8.1%.

FIG. 9c: Graph showing tumor volume for AAV-YFP and AAV-BDNF treated mice.

FIG. 9d: Graph showing tumor weight for AAV-YFP and AAV-BDNF treated mice.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The inventors have discovered that such an enriched environment leads to changes in expression of growth factors and survival of cells within the brain. Moreover, enriched environments have considerable impact on the phenotype of a variety of toxin- and genetically-induced models of human neurological disease. In cancer research, environmental effects have largely focused on diet and exposure to mutagens and carcinogens.

The inventors herein have now determined whether the social and physical components of an animal's environment can have an impact on cancer growth, and if so, what can define any potential mediators. Specifically, the inventors determined whether an enriched environment, one optimized for cerebral health (as defined by improved learning and memory), increased neurogenesis and reduced apoptosis and resistance to external cerebral insults, could also lead to an anti-cancer phenotype—in other words, a mens sana associated with a corpore sano.

One approach to model cancer is the inoculation of malignant cells into their syngeneic rodent hosts. Almost invariably, the cancer cells continue to proliferate following transplantation, leading to solid tumors and ultimately death. One such model is the use of B16 melanoma cells injected into C57BL/6 mice. This model has been useful both in terms of testing a variety of therapeutic strategies, as well as helping to elucidate many aspects of tumor biology.

The following examples are intended to illustrate preferred embodiments of the invention and should not be interpreted to limit the scope of the invention as defined in the claims, unless so specified.

### EXAMPLES

Environmental Enrichment Reduces Tumor Growth

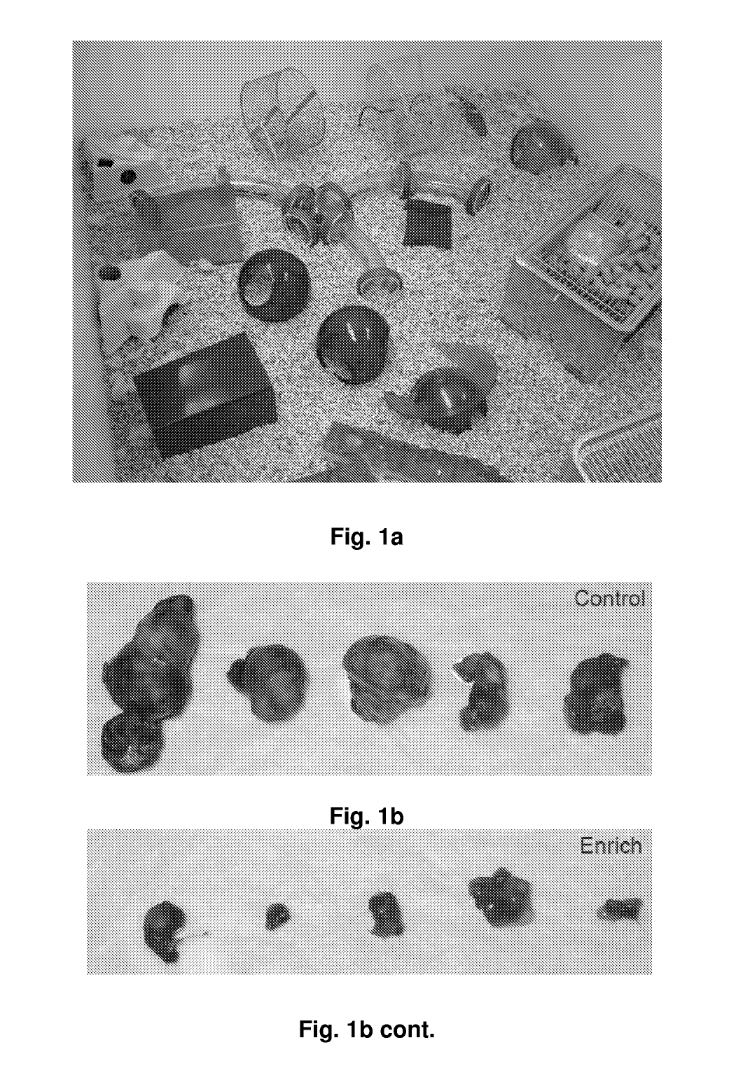

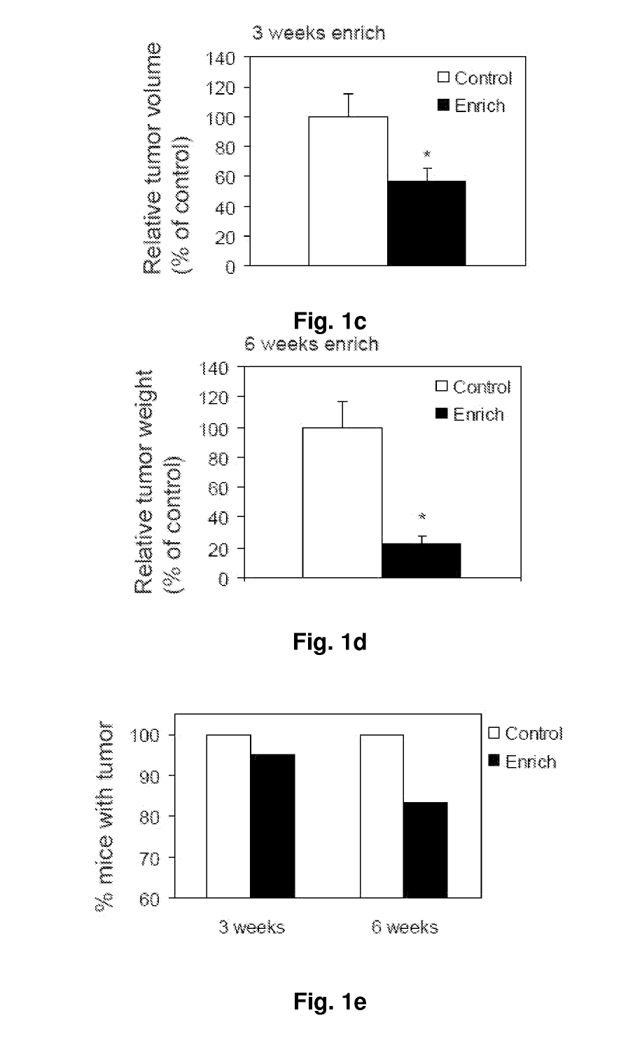

Immediately weaned (three week old) C57BL/6 mice were randomized to live either in an enriched environment (FIG. 1a) or control housing for 3 weeks or 6 weeks (n=18-20 per group).

After 3 or 6 weeks, both enriched and control mice received subcutaneous injections of B16 melanoma cells and were then returned to their respective homes. At 17-19 days post inoculation, the tumor size was determined (FIG. 1b).

In the mice housed in the enriched environment for 3 weeks prior to melanoma transplantation, the mean volume of the tumor was 1155.4+172.4 mm³, 43.1+8.5% smaller than those in the control housing (2031.2+311.1 mm³; P<0.05).

In the 6 week enriched group, the melanoma mass in the enriched animals was 160.6+34.6 mg compared to 703+113.1 mg in the controls, a remarkable 77.2+4.9% reduction in tumor mass (P<0.001). Of particularly interest was that all mice in the control group developed solid tumors; whereas, in the 3 week enriched group 5% of mice had no tumors and in the 6 week enriched animals, this tumor resistant group reached 17% (FIG. 1e).

Enrichment Induces Systemic Metabolic Changes

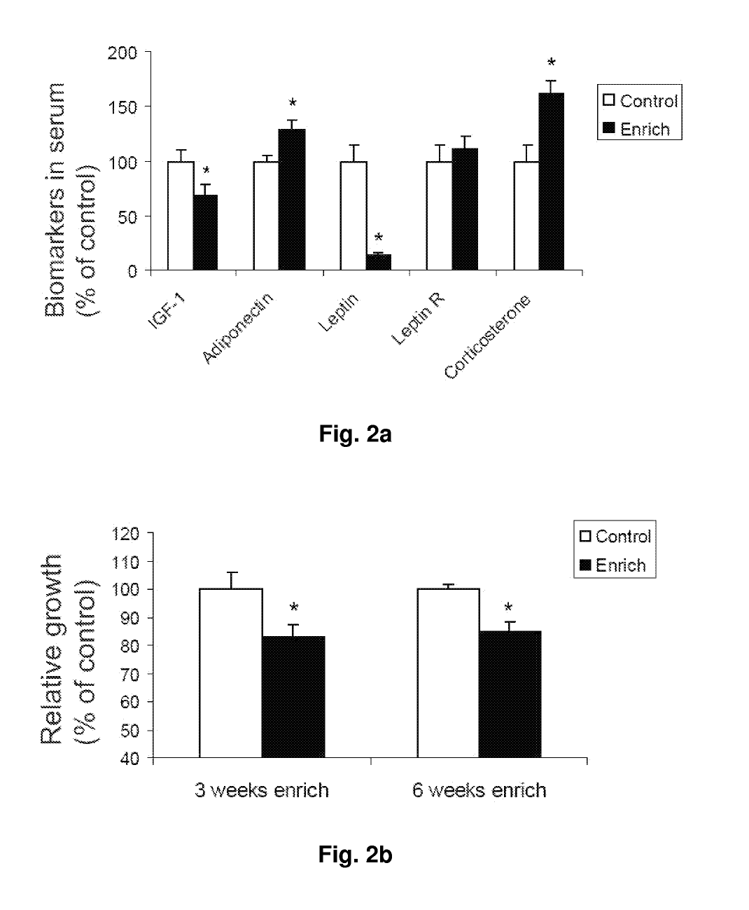

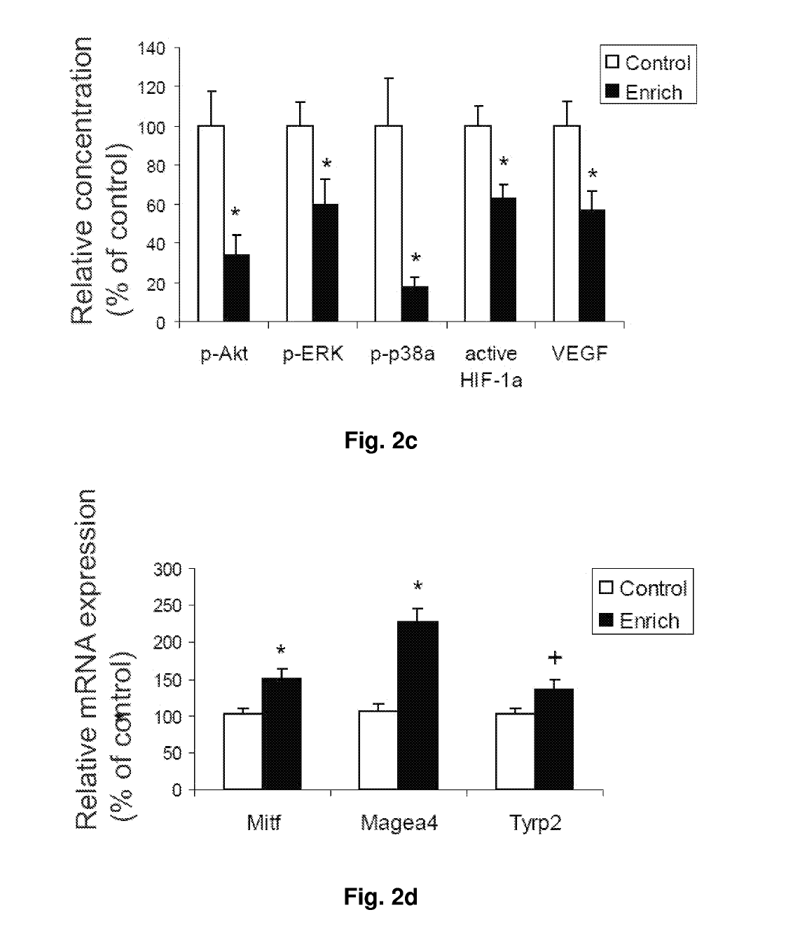

In the mice housed in either control or enriched housing for 6 weeks, peripheral blood was taken prior to inoculation with the melanoma cells. IGF-1 levels have been consistently associated with cancer risk and progression including melanoma. Serum IGF-1 showed a significant reduction in the enriched group (36.1+3.3 ng/ml vs. 24.7+3.5 ng/ml, P<0.05, FIG. 2a) whereas levels of its major binding protein, IGFBP3, did not change significantly (data not shown).

An adipocyte hormone, adiponectin showed a significant increase (FIG. 2a). This abundant protein is of particular interest in diabetes due to its role in regulating insulin sensitivity, but recently it has been shown to have pleiotropic properties including inhibiting angiogenesis in cancer models. The adrenal glucocorticoid and stress hormone, corticosterone was elevated (157.7+10.7 ng/ml vs. 97.1+14.1 ng/ml) (FIG. 2a).

In contrast, serum leptin concentrations in the enriched group was markedly reduced by 87% of control values (236.8+46.7 pg/ml vs. 1778.2+250.3 pg/ml, P<0.01) (FIG. 2a). Leptin is not only the major adipocyte hormone that conveys metabolic information to the brain but is also involved in other pathways including modulating immune interactions. Both leptin-deficient ob/ob mice and leptin receptor-deficient db/db mice have a decreased incidence of spontaneous and oncogene-induced mammary tumors. Of particular relevance is the fact that ob/ob mice have been shown to have reduced B16 melanoma metastasis.

As the serum had a reduction in factors associated with survival and proliferation of cancer cells, the inventors determined whether B16 melanoma cells incubated with either serum obtained from enriched or control housed animals would impact on the growth of these cells in vitro. The sera from both the 3 and 6 week groups of enriched mice significantly slowed the growth of the melanoma cells compared to control sera (FIG. 2b).

Enrichment Influences Signaling Pathways and Gene Expression in the Tumor

Melanoma cells have been associated with activation of a large number of signal transduction enzymes, which can influence the growth of the cancer. For example a genome-wide screen for oncogenes showed reported that 66% of melanoma patients carry an activating mutation in the BRAF gene that leads to constitutive activation of the MAPK pathway. Since aberrant MAPK pathway activation can result in unharnessed cell proliferation, intensive efforts have been made to target MAPK signaling in melanoma. These signal transduction pathways are also regulated in part by extracellular mediators acting via cell surface receptors, and since the enriched environment altered circulating concentrations of growth factors, the inventors herein worked to determine whether downstream signal transduction pathways would be altered in the tumors growing in enriched animals.

The tumors from enriched animals had highly significant decreases in multiple signal transduction pathway mediators including phospho-Akt, phospho-ERK1/ERK2, phospho-p38a (FIG. 2c).

In addition both active HIF-1alpha and vascular endothelial growth factor (VEGF) were also decreased consistent with a reduction in angiogenesis within the tumor (FIG. 2c).

The effects of enrichment, therefore, include a marked shift in the endocrine axis and downstream signaling with changes consistent with an anti-proliferative effect. The inventors also determined whether transcription factors and antigens, specifically associated with the natural history of melanoma were altered by the environmental enrichment paradigm. mRNA was isolated from tumors of both control and enriched animals. Quantitative RT-PCR was used to measure relative expression levels of transcription factors and antigens which have been associated with melanocyte differentiation and progression including microphthalmia-associated transcription factor (Mitf), silver gp100, tyrosinase, tyrosinase related protein 1 and 2 (Tyrp), as well as melanoma antigen family A2 and A4 (Mage). MITF, the key transcription factor regulating the development and differentiation of melanocytes, was significantly elevated in enriched animals, as was the MAGEA4 with a strong trend for an increase in TRYP-2 (FIG. 2d).

MITF leads to differentiation, pigmentation and cell-cycle arrest in melanocytes. Progression of melanoma is associated with decreased differentiation and lower expression of MITF although its function may not be the same in melanoma as in normal melanocytes. The increase in MITF and the genes in its pathway found in enriched animals are consistent with a more differentiated and less progressive tumor as we observed.

Enrichment Enhances Immunocompetence

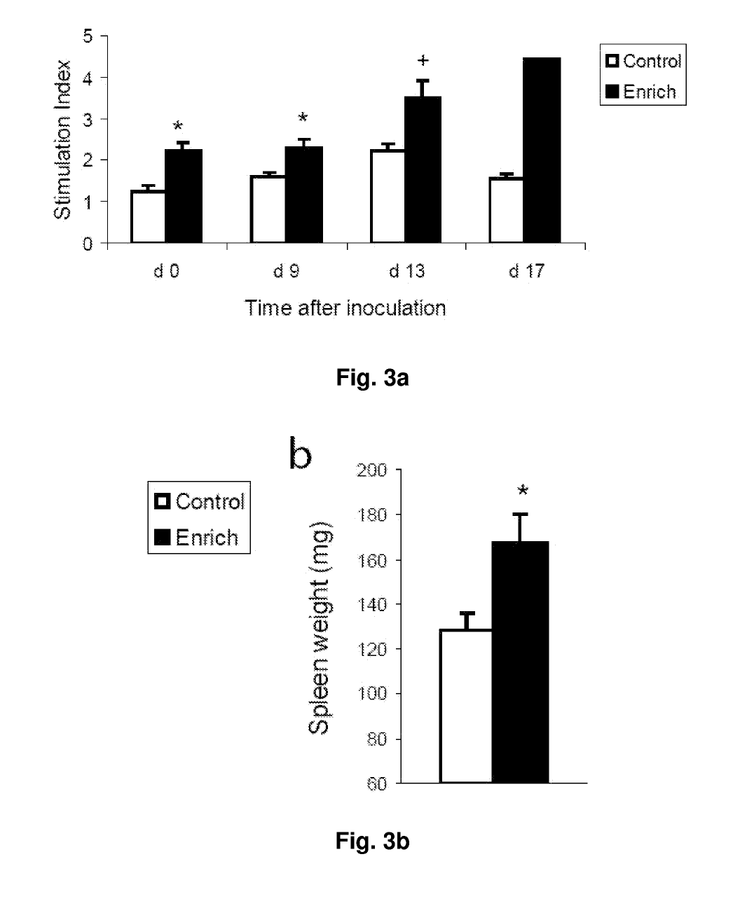

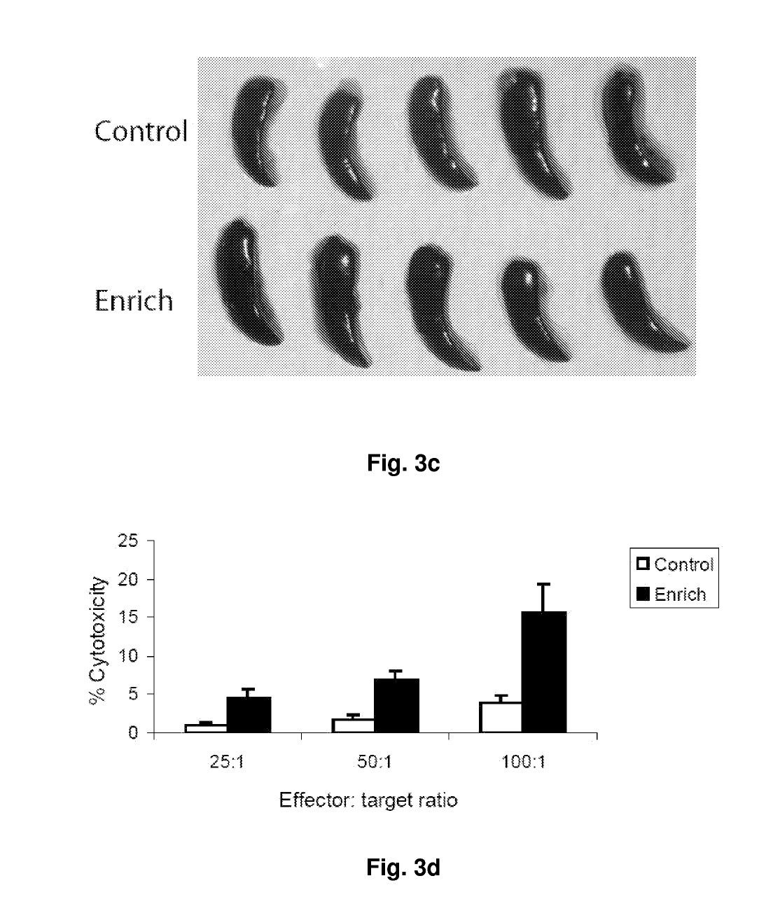

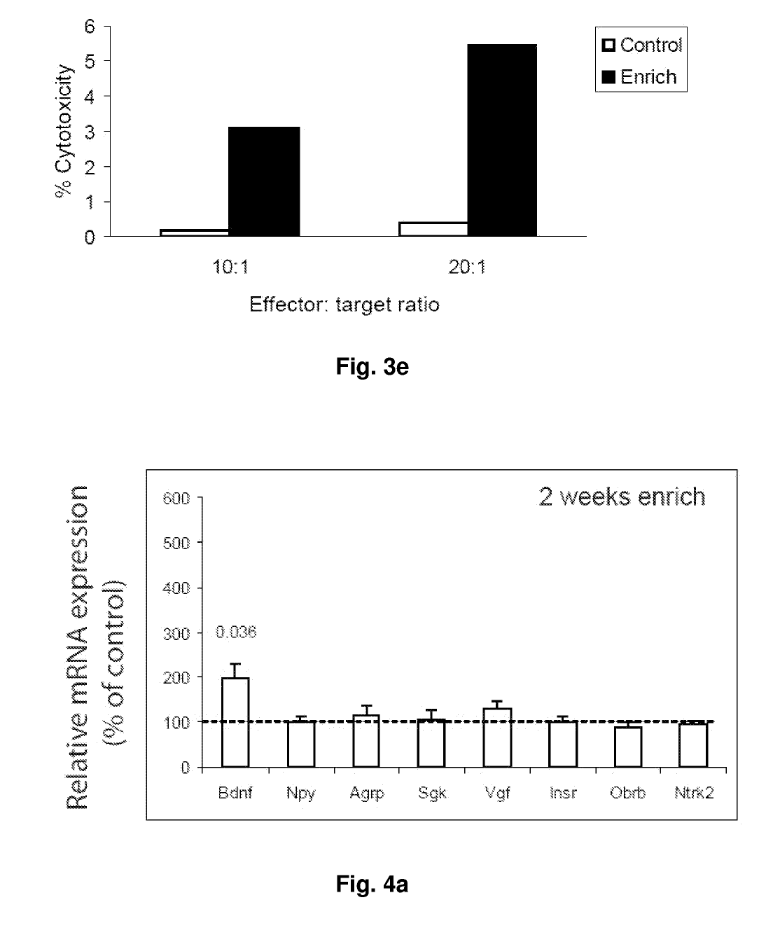

In addition to the profound effects on learning and memory, environmental enrichment may influence immunity directly or indirectly via the interaction between brain, neuroendocrine systems and immune systems. Spleens isolated from enriched animals were significantly enlarged compared to control animals after tumor cells were implanted (FIGS. 3a, 3b).

Furthermore, the splenic lymphocytes of enriched mice showed up to a 2 fold increase of proliferation in response to the T cell mitogen Concavalin A both before (Day 0) and following tumor inoculation (Days 9, 13 & 17) (FIG. 3c).

In addition natural killer cell (NK) activity was greater in enriched mice before tumor inoculation (FIG. 3d), and CD8⁺ T cell cytotoxicity was also increased (FIG. 3e), demonstrating a significant effect on cancer cell specific immunological responses, again consistent with cancer regression.

Enrichment Regulates Hypothalamic Gene Expression

In order to elucidate the mechanisms of this tumor resistance enhanced by enrichment, the inventors targeted the hypothalamus, an area of the brain which is critical in the regulation of both energy balance and neuroendocrine-immune interaction through hypothalamic-pituitary-adrenal (HPA) axis. The hypothalamus contains a number of discrete nuclei including the arcuate (ARC), paraventricular (PVN), ventromedial (VMN), dorsomedial nucleus (DMN) and lateral hypothalamic area (LHA). The circuits regulating energy-homeostasis are found within and connecting these nuclei although other regions of the brain are also important.

The ARC is one of the most important nuclei which contains at least two distinct neuronal populations. One population expresses orexigenic peptides, neuropeptide Y (NPY) and agouti-related protein (AgRP), and the other population expresses the anorexigenic peptides proopiomelanocortin (POMC) and cocaine- and amphetamine-regulated transcript (CART). The ARC is thought to receive information regarding metabolic status from peripheral circulating factors including leptin, insulin, glucose and the gut peptides ghrelin and peptide YY. The neuronal projections from the ARC to other brain areas are thought to mediate the effects of the ARC neuronal system on energy balance.

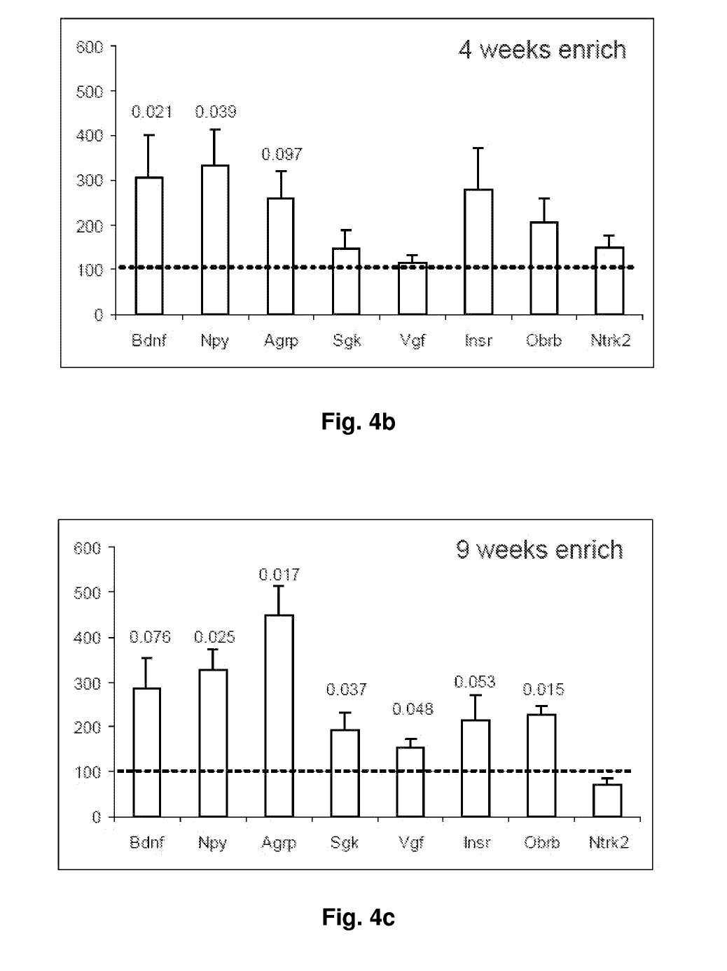

The inventors screened a number of genes known to be involved in metabolic regulation and neuronal-immune crosstalk to evaluate the potential mediators of enrichment-associated metabolic and immune changes. The mice were housed in the enriched or control housing for period of time of 2, 4 and 9 weeks. The ARC was microdissected by laser capture and the mRNA expression was examined by quantitative RT-PCR. At the early time point of 2 weeks enrichment, BDNF was the only gene with significant change, which was upregulated by 2 fold (FIG. 4a).

It remained upregulated at later time points of 4 weeks and 9 weeks. NPY and AgRP expression were increased in mice with 4 weeks enrichment and this upregulation was higher at 9 weeks indicating a stronger response to metabolic changes when exposed to long-term enrichment. Meanwhile, more genes regulating food intake and energy expenditure (serum/glucocorticoid regulated kinase, Sgk and nerve growth factor inducible, Vgf) were upregulated at the long-term enrichment of 9 weeks. In addition, leptin receptor (Obrb) was significantly increased while insulin receptor (Insr) showed a strong trend of upregulation (FIGS. 4a-4c), indicating enhanced sensitivity of ARC neurons to peripheral hormonal signals. In contrast, anorexigenic peptides POMC and CART were not changed in the ARC (data not shown).

Voluntary Wheel Running does not Account for Enrichment-Associated Changes

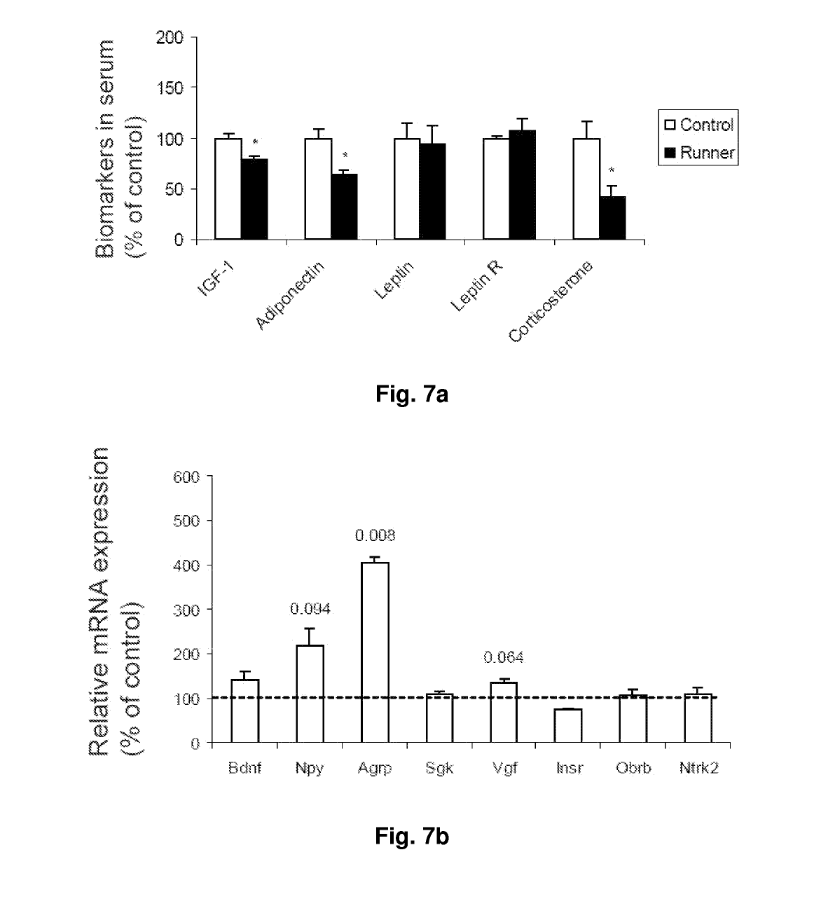

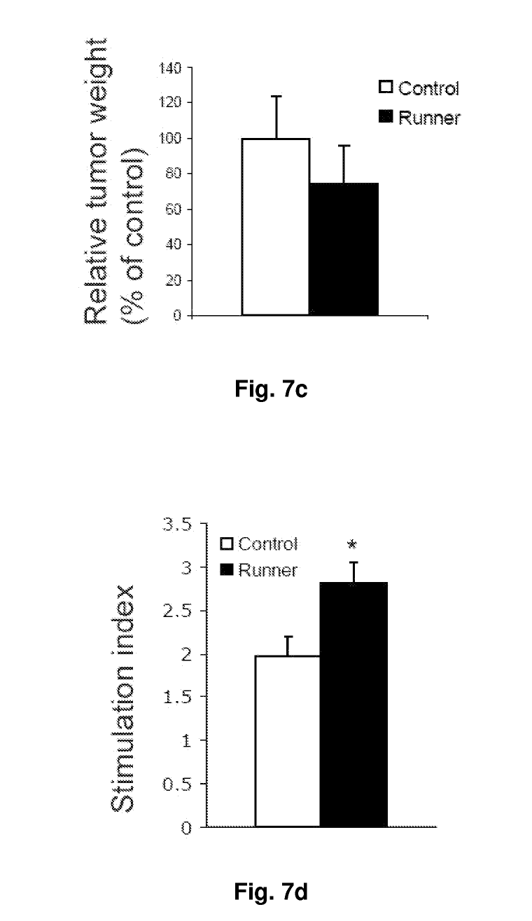

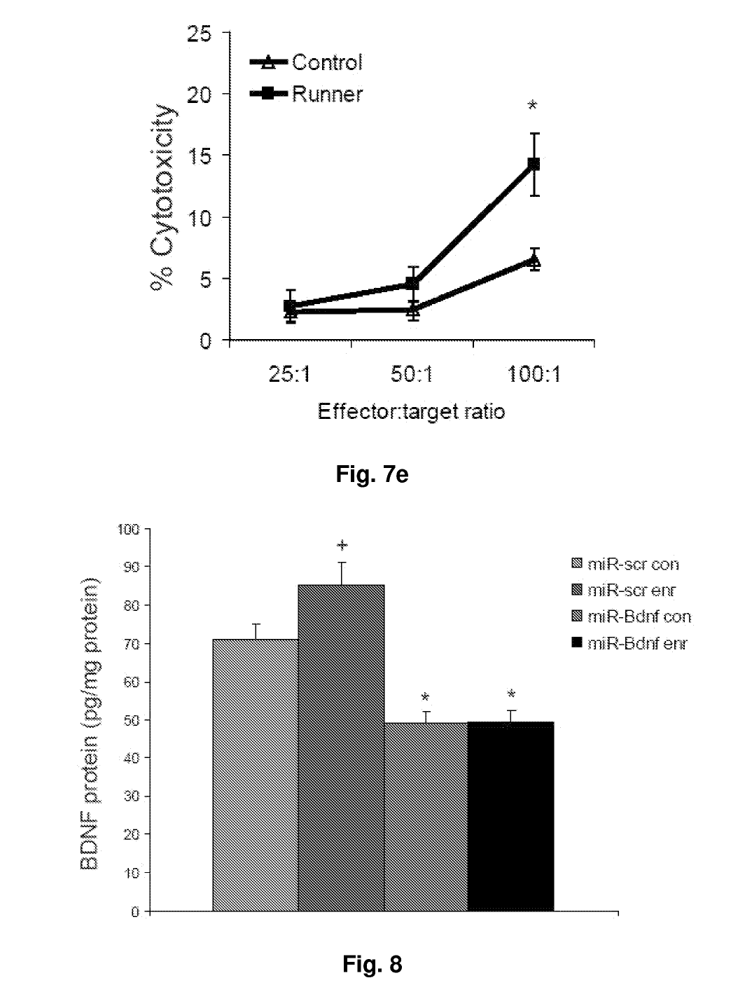

Physical exercise is known to enhance immune function, decrease body fat and recently been shown to inhibit ultraviolet B light-induced carcinogenesis. To investigate whether physical exercise can sufficiently account for the enrichment-induced melanoma resistance, the mice were subjected to voluntary wheel running for 4 weeks followed by tumor implantation. Mice living in cages with free access to running wheels ran approximately 2 km per day. The running led to physiological changes including a trend of reduction of body weight in runners (18.9±0.43 vs. 20.0±0.35, P=0.07) and biomarkers in serum (FIG. 7a) whose pattern was distinctive to the changes of metabolic markers in enrichment mice (FIG. 2a).

In runners, IGF-1 was significantly reduced similar to enrichment; however, the leptin level was not changed while the adiponectin and corticosterone were significantly decreased in contrast to the increase in both factors in the enrichment mice. Although an enhanced immune response was observed in runners (FIGS. 7d, 7e), exercise did not significantly reduce tumor weight in this melanoma model (FIG. 7c). These data suggest physical exercise alone is not sufficient to account for enrichment-induced tumor resistance although it likely contributes. Of particular interest, running affected gene expression in the ARC differently than enrichment.

In contrast to enrichment mice whose BDNF was increased 3 fold at 4 weeks, running of 4 weeks did not upregulate BDNF significantly while the two orexigenic peptides NPY and AgRP were increased (FIG. 7b). Taken together, among the genes being screened, BDNF appeared to be the most selectively responsive to enrichment and is now believed by the inventors herein to be useful as the potential mediator of enrichment-induced tumor resistance, while the change in expression of other genes appeared secondary to systemic physiological changes associated with enrichment.

Hypothalamic Overexpression of BDNF Mimics the Effects of Enrichment on Neuroendocrine, Immune Functions and Tumor Reduction

BDNF has diverse functions in brain development and plasticity, and its expression is highly responsive to activity and environment (at least in the hippocampus). BDNF has been identified recently as an important component of the hypothalamic pathway that controls energy homeostasis. Both peripheral and central administration of BDNF decreased food intake, increased energy expenditure and led to weight loss. Obesity phenotypes have been observed in BDNF heterozygous mice and in a conditional knockout model. It has been shown that BDNF may function downstream of melanocortin-4 receptor which is a critical pathway in the control of body adiposity although the means by which BDNF regulates energy balance remains unclear.

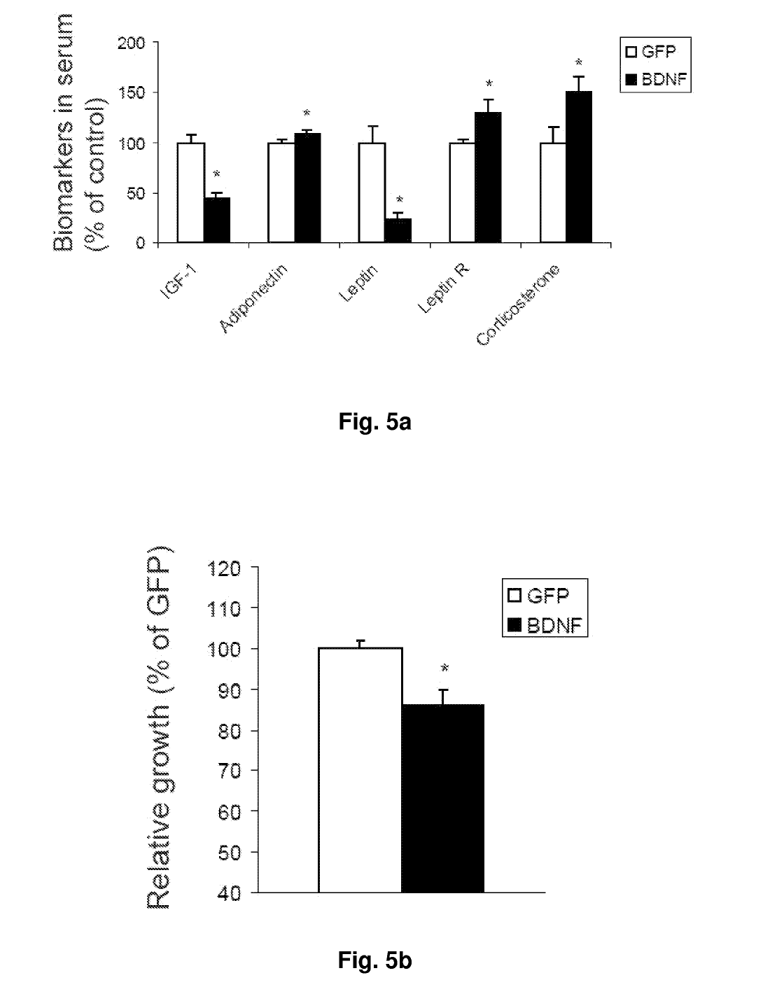

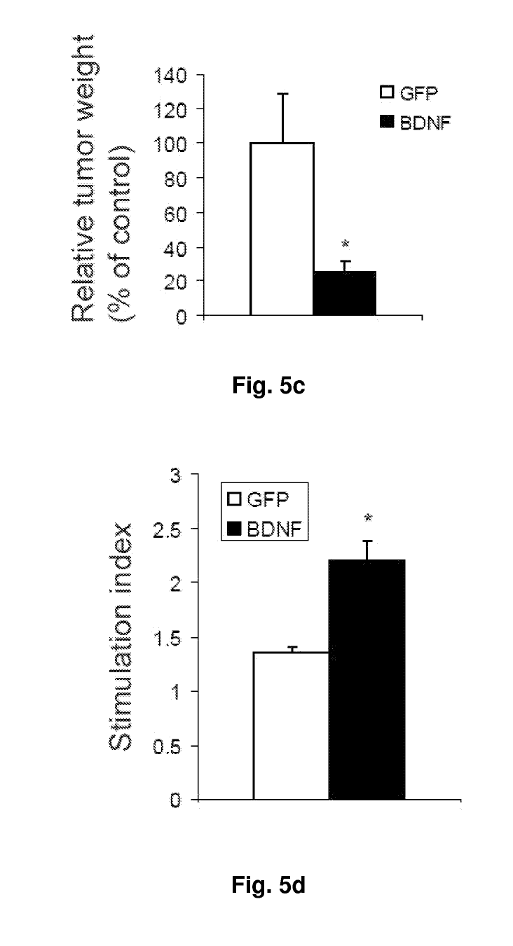

The rAAV serotype 2 vectors were used to deliver human BDNF gene to the ARC bilaterally with a GFP vector as a control. Four weeks after injection, mice receiving BDNF vector showed significantly lower body weight gain than GFP control mice (1.25±0.37 g vs. 4.31±0.44 g, P<0.05). The biomarkers in serum of BDNF mice showed the same pattern of changes (FIG. 5a) as the enrichment mice (FIG. 2a), namely a decrease in IGF-1 and leptin, and an increase in adiponectin and corticosterone.

Leptin circulates in a free form and bound to soluble leptin receptor. Only free leptin is biological active. The soluble leptin receptor concentration was unchanged in enrichment mice (FIG. 2a) whereas it was significantly increased in BDNF mice (FIG. 5a), leading to a reduction in the free leptin index. Consistent with the biomarker changes, B16 melanoma cell growth in vitro was slower when cultured with sera from BDNF mice compared to GFP control (FIG. 5b).

In addition, BDNF mice showed an enhanced immune response (FIG. 5d). Also similar to enrichment mice, tumor weight was significantly decreased in BDNF mice by 75% compared to GFP mice (FIG. 5c).

BDNF Knockdown Inhibits Enrichment-Induced Tumor Resistance

To determine whether knockdown of hypothalamic BDNF could inhibit the tumor resistance and associated physiological changes, a vector expressing microRNA targeting against mouse BDNF (miR-Bdnf) was generated. In vitro experiments demonstrated that this microRNA vector knocked down BDNF mRNA by 65% and protein level by 80%. Also, a control microRNA vector targeting a scrambled sequence (miR-scr) against no known genes was generated.

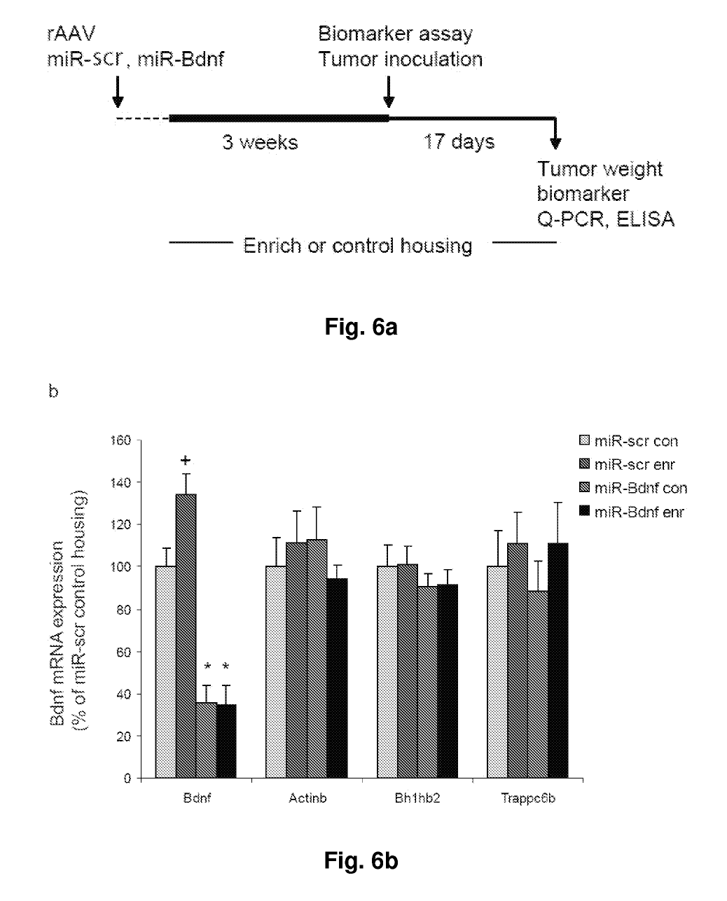

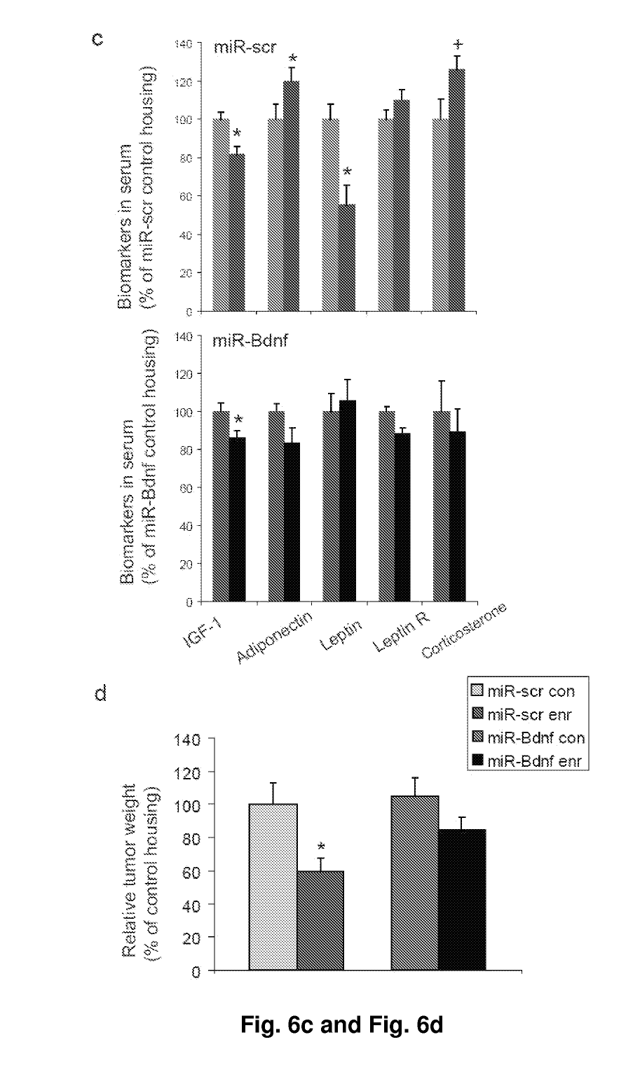

The rAAV serotype 1 vectors of miR-Bdnf or miR-scr were injected bilaterally into the ARC of mice and then assigned them to enriched or standard housing (FIG. 6a). RNAi efficiency in the hypothalamus at both mRNA and protein levels was measured by quantitative RT-PCR and ELISA, respectively.

Both BDNF mRNA (FIG. 6c) and BDNF protein levels (FIG. 8) were reduced significantly in mice receiving miR-Bdnf living in control housing as well as enriched housing compared to mice receiving miR-scr. This approximately 60% reduction of BDNF expression in hypothalamus led to accelerated weight gain in miR-Bdnf mice (191.5% of miR-scr). The specificity of miR-Bdnf was assessed by quantifying the levels of other mRNAs and observed no significant difference in expression of house keeping gene Actb or the genes with sequences most homologous to the targeting sequence of miR-Bdnf Bh1hb or Trappc6b. (FIG. 6b) The changes of biomarkers in serum associated with enrichment was largely preserved in mice receiving miR-scr (FIG. 6c upper panel) while those changes diminished in mice receiving miR-Bdnf except IGF-1 (FIG. 6c lower panel).

After housing the mice in enriched or control housing for 3 weeks, B16 melanoma cells were implanted. Tumor weight was significantly reduced in miR-scr mice living in enriched housing compared to control housing (FIG. 6d, P<0.05). However this enrichment-induced tumor reduction was inhibited in mice receiving miR-Bdnf (FIG. 6d, P=0.113 miR-Bdnf enr. vs. miR-Bdnf con.).

Gene Therapy for Obesity-related Cancer

Increasing epidemiological evidence has demonstrated that obesity is associated with an increased risk of cancer, especially colon cancer.

The inventors herein have now developed several strategies to achieve potent and safe gene therapy for obesity and related metabolic syndromes with AAV-BDNF vectors, including an autoregulatory negative feedback system using RNAi coupled to transgene-induced physiological changes and a definitive knockout via delivery of a second, rescue vector based on Cre-loxP systems. Long-term observation of mice receiving these therapeutic vectors in both diet-induced obesity model and diabetic genetic models showed marked weight loss and alleviation of obesity-associated insulin resistance.

On the other hand, the inventors herein have now shown that environmental enrichment was effective in reducing colon cancer (MC38 colon cancer implant) growth even when the intervention commenced after the cancer was established.

In addition, the inventors herein have demonstrated that hypothalamic BDNF upregulation was a critical component mediating this enrichment-induced tumor inhibition.

The inventors herein now show that hypothalamic gene delivery of AAV-BDNF vector can effectively treat both obesity and cancers associated with obesity, such as colon cancer.

The therapeutic vector can be fine-tuned by combining the autoregulatory strategy (RNAi mimicking physiological negative feedback) with the rescue strategy (knockout via Cre-loxP). Furthermore, in certain embodiments, the miR-Bdnf can be replaced with a miR-WPRE (woodchuck post-transcriptional regulatory element). Unlike BDNF, WPRE does not exist in rodents or humans tissues. It can only be introduced by the gene transfer vectors. Therefore, the microRNA targeting WPRE specifically regulates transgene expression without interference with endogenous genes.

The WPRE seq in the AAV vectors: [SEQ ID NO:4]

Two targeting seqs with the highest scores (Invitrogene RNAi Design Tool) were selected and cloned into the Block-iT PolII miR RNAi expression vector:

[SEQ ID NO: 5]
WPRE 74: CTATGTGGACGCTGCTTTA
[SEQ ID NO: 6]
WPRE155: TCCTGGTTTGTCTCTTTAT

In in vitro experiments, both miR constructs inhibited BDNF expression by at least 90% when co-transfected with the HA-BDNF-WPRE plasmid, as confirmed by ELISA for BDNF. miR-WPRE74 was chosen to construct the autoregulatory plasmid shown below.

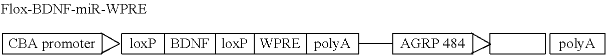

Twenty male C56BL/6 mice were fed with high fat diet (HFD, 45% fat, caloric density 4.73 kcal/g, Research Diets). When body weight reached approximately 40 g, the mice were subcutaneously inoculated with MC38 colon cancer cells (5×10⁴ cells/mouse). Three days after cancer inoculation, mice were randomly assigned to receive rAAV-BDNF (flox-BDNF-miR-WPRE, n=10) or rAAV-YFP (n=10) as control. AAV vector (5×10⁹ particles per site) was injected bilaterally to hypothalamus at the following sites (1.2 mm posterior to the bregma, 0.5 mm lateral to the midline, 6.2 mm dorsal to the bregma). Body weight and tumor size were monitored periodically till sacrifice 21 days after tumor inoculation and 18 days after AAV injection.

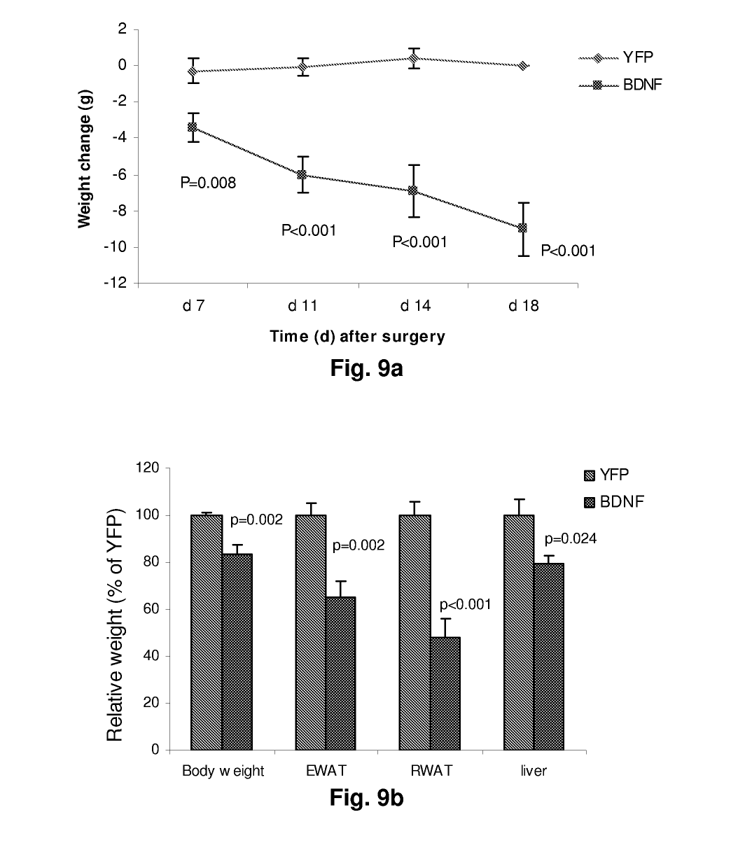

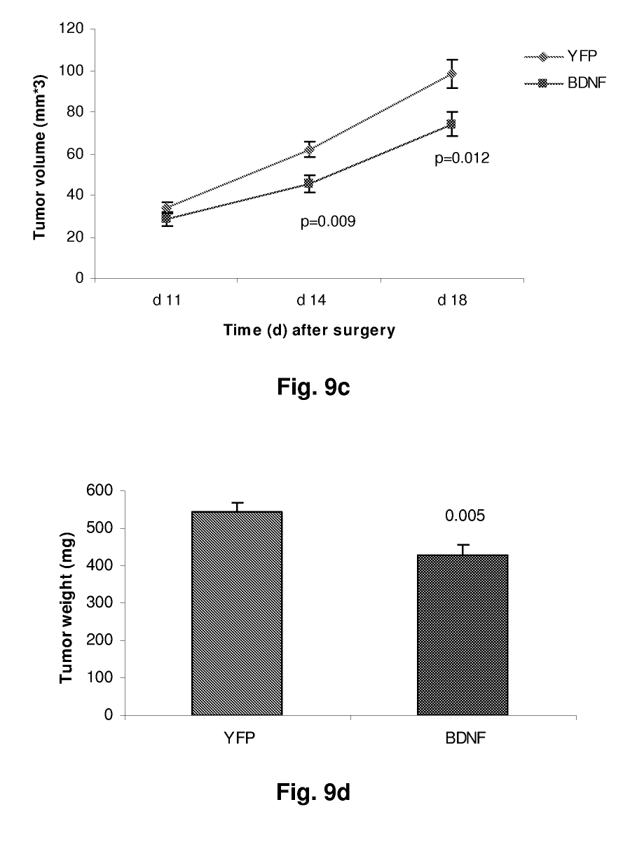

As shown in FIG. 9a, the AAV-BDNF treatment leads to marked weight loss of diet-induced obesity model (DIO) carrying colon cancer.

FIG. 9b shows that AAV-BDNF treatment reduces adiposity particularly white fat. Epididymal white fat pad (EWAT) was reduced by 35.1±7.2% and retroperitoneal white fat pad (RWAT) was reduced by 52.2±8.1%.

AAV-BDNF treatment significantly inhibits colon cancer growth in obese mice as shown by the reduction of tumor volume (FIG. 9c) and tumor weight (FIG. 9d).

These data demonstrate that hypothalamic gene transfer of BDNF can efficiently reverse obesity and reduce colon cancer growth in obese animals.

These data also demonstrate that environmental enrichment can significantly reduce implanted melanoma growth. Moreover, a significant subset of the enriched mice remained tumor free at the end of the experiments while all the controls had tumors. The relative tumor resistance in enrichment mice was associated with improved metabolism and changes in the endocrine axis, which regulates cell proliferation and growth, angiogenesis, as well as enhanced immune responses.

In addition, the expression of melanoma associated genes and several signal transduction pathways were significantly changed in enriched mice. While not wishing to be bound by theory, the inventors herein now believe that all of these may act in concert to inhibit tumor growth. Environmental enrichment represents a complex of physical and social interactions that can influence brain activity, behavior and physiology. The complexity is further manifested in the interaction between central nervous, endocrine and immune systems.

The pathways involved can serve as components of a larger regulatory network that impact on a host's response to cancer. Indeed, physical exercise alone did not account for tumor reduction observed in enrichment although it's likely to serve as an important component. It is unlikely that a single variable accounts for the effects of environmental enrichment, although the inventors herein now believe that changes in the brain could play a central role with the peripheral pathways as the secondary effectors.

BDNF expression in the ARC was highly responsive to environmental stimuli while remaining unchanged by voluntary wheel running, indicating its potential as a mediator underlying mechanisms of this enrichment induced tumor resistance.

The importance of BDNF was assessed using viral vector-mediated hypothalamic overexpression, leading to a phenotype characterized by improved metabolic markers, enhanced immune function and increased resistance to implanted tumor which is similar to those observed in enrichment. Furthermore, knockdown of BDNF expression by RNAi completely blocked the tumor resistance induced by enrichment as well as most of the peripheral changes in metabolism. As such, the inventors herein now believe that hypothalamic BDNF may be a key mediator linking the environment to metabolic and immune functions and tumor growth. The remarkably robust enrichment effect on melanoma growth in vivo can have important implications in cancer therapy. Further, the data show that enriching an individual's interaction with the environment both physically and socially sufficient to alter the endocrine and immunological parameters as measured here, could be combined with other therapies to improve cancer-associated morbidity and mortality.

Methods

Environmental Enrichment Protocol.

For environmental enrichment, male 3 weeks old C57/BL6 mice were housed in groups (18-20 mice per cage) in large cages of 1.5 m×1.5 m×1.0 m supplemented with running wheels, tunnels, igloos, huts, retreats, wood toys, a maze, and nesting material in addition to standard lab chow and water. Control mice were housed under standard laboratory conditions (5 mice per cage). All mice experiments were conducted in compliance with the regulations of the Institutional Animal Ethics Committees.

Melanoma Implantation.

Two environmental enrichment and melanoma implantation paradigms were used.

In paradigm 1, mice were housed in their respective environments for 3 weeks and then subcutaneously implanted a syngeneic melanoma cell line B16F10 on the flank (n=20 per group, 1×10⁵ per mouse). The mice were returned to their respective cages. The tumor volume was measured with caliper and calculated by the formula for ellipsoid (V=length×width²×π/6).

In paradigm 2, the mice were housed in their respective environments for 6 weeks and subcutaneously implanted B16 melanoma cells on the back with the same cell number as paradigm 1 (n=18 per group). The tumors were dissected out from neighboring tissues and measured the weight at the time of sacrifice.

Serum Harvest and Hormone, Growth Factor Measurement.

Blood was collected from retroorbital sinus before implantation of tumor cells and at the day of sacrifice. The mice of each group were anesthetized at the same time with ketamine (87 mg/kg)/xylazine (13 mg/kg) followed by blood withdraw. All blood harvest started at 10:00 am. Serum was prepared by allowing the blood to clot for 30 min on ice followed by centrifugation. Serum was at least diluted 1:5 in serum assay diluent and assayed using the following DuoSet ELISA Development System (R&D Systems): mouse IGF-1, IGFBP-3, Leptin, Leptin R, Adiponectin/Acrp30. Corticosterone was measured using Corticosterone Immunoassay (R&D Systems).

Cell Proliferation.

B16 cells were cultured with RPMI1640 medium plus serum samples from individual mouse of enrichment and control group collected at the time of sacrifice (3 week enrichment paradigm: 5% mouse serum; 6 week enrichment paradigm: 3% mouse serum, n=9-10 in each group). Proliferation was measured using CellTiter 96A_{quesous} One Solution Cell Proliferation Assay (Promega).

Signal Transduction Assay.

Nuclear proteins were extracted from dissected tumors using CelLytic NuCLEAR Extraction Kit (Sigma). Protein concentration was measured with BCA Protein Assay (Pierce). 37.5 μg nuclear protein/well was assessed for active HIF-1α and active STAT3 (Human/Mouse Active HIF-1α Activity Assay, Active STAT3 Activity Assay, R&D Systems, n=7 in each group). The VEGF in the cytoplasmic extraction from the same tumor sample was measured using mouse VEGF DuoSet ELISA (R&D). Additional tumor lysates were prepared using the Lysis buffer according to the manufacturer's instruction and measured the phospho-Akt1 (S473), phospho-ERK1/ERK2. phospho-p38α (T180/Y182), phospho-JNK (Pan) using Intracellular DuoSet IC (R&D Systems, n=7 in each group).

Quantification of Gene Expression in Tumor.

Tumors were dissected from mice undergone 6 week-enrichment paradigm (n=8 in each group) and isolated total RNA using RNeasy Mini Kit plus RNase-free DNase treatment (Qiagen). First-strand cDNA were generated using TaqMan Reverse Transcription Reagent (Applied Biosystems) and carried out quantitative PCR using ABI PRISM 7000 Sequence Detection System with the Power SYBR Green PCR Master Mix (Applied Biosystems). Primers were designed to detect the following mouse mRNA: Mitf, Magea2, Magea4, Si, Tyr, Tyrp1, Tyrp2.

The data were calibrated to endogenous control Actb and quantified the relative gene expression using the equation T₀/R₀=K×2^{(CT,R-CT,T)}. T₀ is the initial number of target gene mRNA copies, R₀ is the initial number of internal control gene mRNA copies, CT,T is the threshold cycle of the target gene, CT,R is the threshold cycle of the internal control gene and K is a constant.

Splenocyte Proliferation Assay.

Splenocytes were harvested from mice of the 6 week-enrichment group at several time points: before tumor inoculation and d 9, d 13 and d 17 after tumor cell inoculation (n=5 in each group at each time point). Single-cell splenocyte suspensions were prepared by teasing spleens and passing through 40 μm Cell Strainer. Erythrocytes were depleted with Red Blood Cell Lysis Buffer (Sigma). Splenocytes were washed 3 times and the viability was assessed by Trypan blue exclusion (usually >90%). Splenocytes were seeded in 96-well plate in complete medium (RPMI1640, 25 mM HEPES, 2 mM L-glutamine, 50 μM β-mercaptoethanol, 2 g/L sodium bicarbonate, 5% FBS). Quadruplicate of cells from each mouse spleen were stimulated with 0 μg/ml of mitogen or 5 μg/ml Concanavalin-A (Sigma) and cultured for 48-72 hrs. Cell proliferation was determined using CellTiter 96A_{quesous} One Solution Cell Proliferation Assay (Promega). Data were expressed as Stimulation Index=mean OD of wells with Concanavalin-A stimulation/mean OD of the wells without stimulation.

CD8 T Cell Preparation.

Highly enriched CD8 T cells were prepared from splenocytes using Murine T Cell CD8 Subset Column Kit (R&D Systems) according to the manufacturer's instruction. Splenocytes from 4 spleens of each group were pooled to prepared CD8 T cells.

Cell-Mediated Cytotoxicity Assay.

Immune cell cytotoxicity was assayed using The CytoTox96 Assay (Promega) according to the manufacturer's instruction. For NK cell activity, splenocytes were prepared from mice undergone 6 week-enrichment. When splenocytes were incubated with B16 cells at various effector: target ratio, the effector NK cells lysed the target cells and the LDH release was measured. Each reaction was performed in quadruplicate. The data were calculated using the following formula: % cytotoxicity=(Experimental release−Effector spontaneous release−Target spontaneous release)/(Target maximum release−Target spontaneous release)×100. Similar assay was performed to measure the cytotoxicity of the CD8 T cell population as described herein.

Microdissection of Arcuate Nucleus with Laser Capture.

Mice of 3 weeks of age were randomized to live in standard housing or enriched housing for 2, 4 and 9 weeks. At each time point, brains were isolated and stored at −80° C. until microdis section. Brain sections were cut at 16 μm thickness and mounted the sections onto RNase-free membrane-coated glass slides (PALM MembraneSlides; P.A.L.M. Microlaser Technologies). Sections were dehydrated in cold 80% ethanol for 5 minutes, then successively dipped in Cresyl violet (0.1% in 100% ethanol), 75% alcohol, 100% alcohol. For each animal, approximately 35 sections were collected using a PALM Laser capture microscope (P.A.L.M. Microlaser Technologies). Samples were collected onto the cap of an eppendorf tube filled with 200 lysis buffer and rapidly frozen at −80° C. Total RNAs were prepared from the arcuate nucleus tissues using RNeasy Micro kit (Qiagene).

Voluntary Running Experiment.

30 male 3 weeks old C57/BL6 mice were randomized to 2 groups: control mice housed under standard laboratory conditions (5 mice per cage, n=14) and runners housed in cages with free access to running wheel (Mouse activity wheel with plastic home cage, Med Associates), 4 mice per running cage (n=16). Given the difficulty for the 3 weeks old mice to use the activity wheel attached to the home, small plastic running wheel were put in the home cage for 2 weeks and then removed when the mice were ready to use the attached activity wheel. Running activity was recorded for 2 weeks. After being housed in the running cages for 4 weeks, mice were inoculated with B16 melanoma cells as described herein and continued to stay in running cages or standard cages till sacrifice.

rAAV Vector Construction and Packaging.

The rAAV plasmid contains a vector expression cassette consists of the CMV enhancer and chicken β-actin promoter, WPRE and bovine growth hormone poly-A flanked by AAV inverted terminal repeats. Human BDNF cDNA was fused to HA tag at the 5′ terminal and then inserted to the multiple cloning sites between the CBA promoter and WPRE sequence. EGFP was cloned to the rAAV plasmid as control. rAAV serotype 2 vectors were packaged, purified and the vectors were adjusted to 2×10¹³ genomic particles per ml in PBS for injection.

AAV Mediated BDNF Overexpression.

26 C57BL/6 mice, male, 8 weeks of age, were randomly assigned to receive AAV-BDNF (n=10) or AAV-GFP (n=16). rAAV vectors (1×10¹⁰ genomic particles per site) were injected bilaterally into the arcuate hypothalamic nuclei (ARC) at the stereotaxic coordinates −1.2 AP, ±0.5 ML, −6.2 DV (mm from bregma) using a microinfusion pump. Four weeks after AAV injection, blood was drawn and the mice were with inoculated B16 melanoma.

microRNA Vector Construction and AAV1 Vector Production.

microRNA were used to target mouse BDNF. Two targeting sequences in the BDNF coding sequence were cloned into Block-iT PolII miR RNAi expression vector (pcDNA6.2-Gw/miR, Invitrogen). In in vitro experiments, both miR constructs inhibited BDNF expression when co-transfected with a BDNF expression plasmid confirmed by Q-PCR and BDNF ELISA.

The miR-BDNF921 (mature miR seq: AAGTGTACAAGTCCGCGTCCT) [SEQ ID NO: 1] was chosen for in vivo experiment. This miR-Bdnf and a miR-scramble (miR-scr, with scramble sequence targeting no known gene, Invitrogen) were subcloned to the AAV plasmid as described above. AAV serotype 1 vectors were generated and adjusted the vectors to 2×10¹³ genomic particles per ml in PBS for injection.

AAV-microRNA Experiment.

7-weeks-old C57/BL6 mice were randomly assigned to received AAV-miR-Bdnf (n=20) or AAV-miR-scr (n=20). 0.7 μl of AAV vectors (1.4×10¹⁰ particles) were injected bilaterally into the ARC at the stereotaxic coordinates described herein. Ten days after surgery, the mice were split into four groups and exposed to enriched (n=10 per AAV vector) or control (n=10 per AAV vector) housing as described herein. After 3 weeks in enriched or control housing, blood was drawn and the mice were and inoculated with B16 melanoma cells; the mice were kept in the respective housing till sacrifice.

BDNF Expression Quantification.

Hypothalamus was dissected and prepared total RNA from half of the hypothalamic tissue and subjected it to quantitative RT-PCR. The data of quantitative RT-PCR were calibrated to the endogenous control gene Eef2. Lystates were prepared from the other half of the hypothalamic tissue and measured BDNF protein level using ELISA (BDNF Emax ImmunoAssay System, Promega). The BDNF protein level was calibrated to total protein level.

Statistical Analysis.

Values are expressed as mean±s.e.m. One-way ANOVA was used to analyze tumor volume, tumor weight, ELISA and B16 proliferation. Multivariate ANOVA was used to analyze quantitative RT-PCR data. For the immune cell proliferation and cytotoxicity, the overall significance was determined using repeated measures ANOVA.

### EXAMPLES OF USES

### Pharmaceuticals

Pharmaceutical compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, modulators, or drugs (e.g., antibiotics). In particular embodiments, the pharmaceutical compositions also contain a pharmaceutically acceptable carrier or excipient. Such materials should be non-toxic and should not interfere with the efficacy of the active ingredient. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences [Mack Pub. Co., 18th Edition, Easton, Pa. (1990)]. The precise nature of the carrier or other material may depend on the route of administration. As described herein, the present invention is directed to administering the expression vectors and compositions thereof of the invention to target cells in the nervous system.

In accordance with the present invention, an expression vector comprising BDNF that is to be given to an individual, is administered preferably in a “therapeutically effective amount” or a “prophylactically effective amount” (as the case may be, although prophylaxis may be considered therapy), this being sufficient to show benefit to the individual.

Although the compositions of the invention have been described with respect to human therapeutics, it will be apparent to one skilled in the art that these tools are also useful in animal experimentation directed to developing treatment regimens for animal subjects that have a neurological disorder. Indeed, as described herein, animal subjects which exhibit symptoms characteristic of various disorders have been developed that serve as model systems for human disorders. As such, the examples herein are provided to illustrate certain embodiments of the invention. They are not intended to limit the invention in any way.

### DEFINITIONS

Various terms relating to the molecules and methods of the present invention are used hereinabove and also throughout the specifications and claims.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote.

With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

A “vector” is a replicon, such as plasmid, phage, cosmid, or virus into which another nucleic acid segment may be operably inserted so as to bring about the replication or expression of the segment. The term “vectors” can refer to vectors that can be delivered to the target cells by using viral vectors or by using non-viral vectors. In certain embodiments, the invention uses adeno-associated viral vectors comprising a nucleotide sequence encoding BDNF for gene delivery. The vectors can be constructed using known techniques to provide at least the operatively linked components of control elements including a transcriptional initiation region, an exogenous nucleic acid molecule, a transcriptional termination region and at least one post-transcriptional regulatory sequence. The control elements are selected to be functional in the targeted cell. The resulting construct which contains the operatively linked components is flanked at the 5′ and 3′ region with functional AAV ITR sequences. The skilled artisan can appreciate that regulatory sequences can often be provided from commonly used promoters derived from viruses. Use of viral regulatory elements to direct expression of the protein can allow for high level constitutive expression of the protein in a variety of host cells. Alternatively, the regulatory sequences of the vector can direct expression of the gene preferentially in a particular cell type, i.e., tissue-specific regulatory elements can be used. In certain embodiments, the promoter is tissue specific and is essentially not active outside the target cells, or the activity of the promoter is higher in the target cells than in other systems. Suitable host cells for producing recombinant particles include, but are not limited to, microorganisms, yeast cells, insect cells, and mammalian cells, that can be, or have been, used as recipients of a exogenous nucleic acid molecule. Thus, a “host cell” as used herein generally refers to a cell which has been transfected with an exogenous nucleic acid molecule. The host cell includes any eukaryotic cell or cell line so long as the cell or cell line is not incompatible with the protein to be expressed, the selection system chosen or the fermentation system employed.

An “expression vector” is a vector which (due to the presence of appropriate transcriptional and/or translational control sequences) is capable of expressing a DNA molecule which has been cloned into the vector and of thereby producing an RNA or protein product encoded by an expressible gene provided by said DNA. Expression of the cloned sequences occurs when the expression vector is introduced into an appropriate host cell. If a prokaryotic expression vector is employed, then the appropriate host cell would be any prokaryotic cell capable of expressing the cloned sequences. Similarly, when a eukaryotic expression vector is employed, e.g., for genetic manipulation prior to gene delivery, then the appropriate host cell would be any eukaryotic cell capable of expressing the cloned sequences.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like. The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on to or inherited by progeny cells or organisms of the recipient cell or organism. In other applications, the introduced nucleic acid may exist in the recipient cell or host organism only transiently.

The term “substantially pure” refers to a preparation comprising at least 50-60% by weight of the compound of interest (e.g., nucleic acid, oligonucleotide, protein, etc.). More preferably, the preparation comprises at least 75% by weight, and most preferably 90-99% by weight, of the compound of interest. Purity is measured by methods appropriate for the compound of interest (e.g. chromatographic methods, agarose or polyacrylamide gel electrophoresis, HPLC analysis, and the like).

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

The term “immune response” refers to a physiological response of a subject which is triggered by an antigen or antigenic agent, whereby the humoral branch (relating generally to activation of B cells and the generation of immunologically specific antibodies) and/or the cellular branch (pertaining generally to T cell mediated responses) of the immune system are activated.

As used herein, the terms “reporter,” “reporter system”, “reporter gene,” or “reporter gene product” shall mean an operative genetic system in which a nucleic acid comprises a gene that encodes a product which when expressed produces a reporter signal that is readily measurable, e.g., by biological assay, immunoassay, radioimmunoassay, or by colorimetric, fluorogenic, chemiluminescent or other method. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, and may include, but not be limited to, such elements as promoters, enhancers, translational control sequences, poly A addition signals, transcriptional termination signals and the like.

The term “percent identical” is used herein with reference to comparisons among nucleic acid or amino acid sequences. Nucleic acid and amino acid sequences are often compared using computer programs that align sequences of nucleic or amino acids thus defining the differences between the two. For purposes of this invention comparisons of nucleic acid sequences are performed using the GCG Wisconsin Package version 9.1, available from the Genetics Computer Group in Madison, Wis. For convenience, the default parameters (gap creation penalty=12, gap extension penalty=4) specified by that program are intended for use herein to compare sequence identity. Alternately, the Blastn 2.0 program provided by the National Center for Biotechnology Information (at http://www.ncbi.nim.nih.gov/blast/; Altschul et al., 1990, J Mol Biol 215:403-410) using a gapped alignment with default parameters, may be used to determine the level of identity and homology between nucleic acid sequences and amino acid sequences.

The term “functional” as used herein implies that the nucleic or amino acid sequence is functional for the recited assay or purpose.

The term “functional fragment” as used herein refers to a portion or sub domain of polypeptide or peptide that retains an activity of the full length polypeptide or peptide. With respect to BDNF, a functional fragment of BDNF is a portion or sub domain of a BDNF peptide that retains an activity of BDNF. In the context of the present invention, an activity of BDNF may refer, as described herein, to the ability of BDNF to ameliorate symptoms associated with a tumor associated disorder.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic subjects such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The term “central nervous system” or “CNS” as used herein refers to the art recognized use of the term. The CNS pertains to the brain, cranial nerves and spinal cord. The CNS also comprises the cerebrospinal fluid, which fills the ventricles of the brain and the central canal of the spinal cord.

The term “modifies” or “modified” are used interchangeably herein and refer to the up-regulation or down-regulation of a target gene or a target protein. The term modifies or modified also refers to the increase, decrease, elevation, or depression of processes or signal transduction cascades involving a target gene or a target protein. Modification to the concentrations may occur when a therapeutic agent, alters the concentration. For example, modifications that result in an increase BDNF concentration by the expression of BDNF. Modifications can also result from the addition of a therapeutic agent that inactivates BDNF. The effect is to block the degradation of BDNF and thereby increase its concentration. The term modifies also includes increasing, or activating BDNF with therapeutic agents that activate BDNF. Non-limiting examples of modifications includes modifications of morphological and functional processes, under- or over production or expression of a substance or substances.

The term “tissue-specific promoter” as used herein refers to a promoter that is operable in target cells In certain embodiments, the promoter is tissue specific and is essentially not active outside the target cells, or the activity of the promoter is higher in the target cells that in other systems.

Further, while the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

```
BDNF (miR-Bdnf)
[SEQ ID NO: 1]
AAGTGTACAAGTCCGCGTCCT
BDNF brain-derived neurotrophic factor NM_170735,
Accession Numbers AB038670,
[SEQ ID NO: 2]
MTILFLTMVISYFGCMKAAPMKEANIRGQGGLAYPGVRTHGTLE
SVNGPKAGSRGLTSLADTFEHVIEELLDEDQKVRPNEENNKDADLYTSRVMLSSQVPL
EPPLLFLLEEYKNYLDAANMSMRVRRHSDPARRGELSVCDSISEWVTAADKKTAVDMS
GGTVTVLEKVPVSKGQLKQYFYETKCNPMGYTKEGCRGIDKRHWNSQCRTTQSYVRAL
TMDSKKRIGWRFIRIDTSCVCTLTIKRGR
[SEQ ID NO: 3]
      1 gttccccaac tgctgtttta ttgtgctatt catgcctaga catcacatag ctagaaaggc
    61 ccatcagacc cctcaggcca ctgctgttcc tgtcacacat tcctgcaaag gaccatgttg
  121 ctaacttgaa aaaaattact attaattaca cttgcagttg ttgcttagta acatttatga
  181 ttttgtgttt ctcgtgacag catgagcaga gatcattaaa aattaaactt acaaagctgc
  241 taaagtggga agaaggagaa cttgaagcca caatttttgc acttgcttag aagccatcta
  301 atctcaggtt tatatgctag atcttggggg aaacactgca tgtctctggt ttatattaaa
  361 ccacatacag cacactactg acactgattt gtgtctggtg cagctggagt ttatcaccaa
  421 gacataaaaa aaccttgacc ctgcagaatg gcctggaatt acaatcagat gggccacatg
  481 gcatcccggt gaaagaaagc cctaaccagt tttctgtctt gtttctgctt tctccctaca
  541 gttccaccag gtgagaagag tgatgaccat ccttttcctt actatggtta tttcatactt
  601 tggttgcatg aaggctgccc ccatgaaaga agcaaacatc cgaggacaag gtggcttggc
  661 ctacccaggt gtgcggaccc atgggactct ggagagcgtg aatgggccca aggcaggttc
  721 aagaggcttg acatcattgg ctgacacttt cgaacacgtg atagaagagc tgttggatga
  781 ggaccagaaa gttcggccca atgaagaaaa caataaggac gcagacttgt acacgtccag
  841 ggtgatgctc agtagtcaag tgcctttgga gcctcctctt ctctttctgc tggaggaata
  901 caaaaattac ctagatgctg caaacatgtc catgagggtc cggcgccact ctgaccctgc
  961 ccgccgaggg gagctgagcg tgtgtgacag tattagtgag tgggtaacgg cggcagacaa
1021 aaagactgca gtggacatgt cgggcgggac ggtcacagtc cttgaaaagg tccctgtatc
1081 aaaaggccaa ctgaagcaat acttctacga gaccaagtgc aatcccatgg gttacacaaa
1141 agaaggctgc aggggcatag acaaaaggca ttggaactcc cagtgccgaa ctacccagtc
1201 gtacgtgcgg gcccttacca tggatagcaa aaagagaatt ggctggcgat tcataaggat
1261 agacacttct tgtgtatgta cattgaccat taaaagggga agatagtgga tttatgttgt
1321 atagattaga ttatattgag acaaaaatta tctatttgta tatatacata acagggtaaa
1381 ttattcagtt aagaaaaaaa taattttatg aactgcatgt ataaatgaag tttatacagt
1441 acagtggttc tacaatctat ttattggaca tgtccatgac cagaagggaa acagtcattt
1501 gcgcacaact taaaaagtct gcattacatt ccttgataat gttgtggttt gttgccgttg
1561 ccaagaactg aaaacataaa aagttaaaaa aaataataaa ttgcatgctg ctttaattgt
1621 gaattgataa taaactgtcc tctttcagaa aacagaaaaa aaacacacac acacacaaca
1681 aaaatttgaa ccaaaacatt ccgtttacat tttagacagt aagtatcttc gttcttgtta
1741 gtactatatc tgttttactg cttttaactt ctgatagcgt tggaattaaa acaatgtcaa
1801 ggtgctgttg tcattgcttt actggcttag gggatggggg atggggggta tatttttgtt
1861 tgttttgtgt ttttttttcg tttgtttgtt ttgtttttta gttcccacag ggagtagaga
1921 tggggaaaga attcctacaa tatatattct ggctgataaa agatacattt gtatgttgtg
1981 aagatgtttg caatatcgat cagatgacta gaaagtgaat aaaaattaag gcaactgaac
2041 aaaaaaatgc tcacactcca catcccgtga tgcacctccc aggccccgct cattctttgg
2101 gcgttggtca gagtaagctg cttttgacgg aaggacctat gtttgctcag aacacattct
2161 ttccccccct ccccctctgg tctcctcttt gttttgtttt aaggaagaaa aatcagttgc
2221 gcgttctgaa atattttacc actgctgtga acaagtgaac acattgtgtc acatcatgac
2281 actcgtataa gcatggagaa cagtgatttt tttttagaac agaaaacaac aaaaaataac
2341 cccaaaatga agattatttt ttatgaggag tgaacatttg ggtaaatcat ggctaagctt
2401 aaaaaaaact catggtgagg cttaacaatg tcttgtaagc aaaaggtaga gccctgtatc
2461 aacccagaaa cacctagatc agaacaggaa tccacattgc cagtgacatg agactgaaca
2521 gccaaatgga ggctatgtgg agttggcatt gcatttaccg gcagtgcggg aggaatttct
2581 gagtggccat cccaaggtct aggtggaggt ggggcatggt atttgagaca ttccaaaacg
2641 aaggcctctg aaggaccctt cagaggtggc tctggaatga catgtgtcaa gctgcttgga
2701 cctcgtgctt taagtgccta cattatctaa ctgtgctcaa gaggttctcg actggaggac
2761 cacactcaag ccgacttatg cccaccatcc cacctctgga taattttgca taaaattgga
2821 ttagcctgga gcaggttggg agccaaatgt ggcatttgtg atcatgagat tgatgcaatg
2881 agatagaaga tgtttgctac ctgaacactt attgctttga aactagactt gaggaaacca
2941 gggtttatct tttgagaact tttggtaagg gaaaagggaa caggaaaaga aaccccaaac
3001 tcaggccgaa tgatcaaggg gacccatagg aaatcttgtc cagagacaag acttcgggaa
3061 ggtgtctgga cattcagaac accaagactt gaaggtgcct tgctcaatgg aagaggccag
3121 gacagagctg acaaaatttt gctccccagt gaaggccaca gcaaccttct gcccatcctg
3181 tctgttcatg gagagggtcc ctgcctcacc tctgccattt tgggttagga gaagtcaagt
3241 tgggagcctg aaatagtggt tcttggaaaa atggatcccc agtgaaaact agagctctaa
3301 gcccattcag cccatttcac acctgaaaat gttagtgatc accacttgga ccagcatcct
3361 taagtatcag aaagccccaa gcaattgctg catcttagta gggtgaggga taagcaaaag
3421 aggatgttca ccataaccca ggaatgaaga taccatcagc aaagaatttc aatttgttca
3481 gtctttcatt tagagctagt ctttcacagt accatctgaa tacctctttg aaagaaggaa
3541 gactttacgt agtgtagatt tgttttgtgt tgtttgaaaa tattatcttt gtaattattt
3601 ttaatatgta aggaatgctt ggaatatctg ctatatgtca actttatgca gcttcctttt
3661 gagggacaaa tttaaaacaa acaacccccc atcacaaact taaaggattg caagggccag
3721 atctgttaag tggtttcata ggagacacat ccagcaattg tgtggtcagt ggctctttta
3781 cccaataaga tacatcacag tcacatgctt gatggtttat gttgacctaa gatttatttt
3841 gttaaaatct ctctctgttg tgttcgttct tgttctgttt tgttttgttt tttaaagtct
3901 tgctgtggtc tctttgtggc agaagtgttt catgcatggc agcaggcctg ttgctttttt
3961 atggcgattc ccattgaaaa tgtaagtaaa tgtctgtggc cttgttctct ctatggtaaa
4021 gatattattc accatgtaaa acaaaaaaca atatttattg tattttagta tatttatata
4081 attatgttat tgaaaaaaat tggcattaaa acttaaccgc atcagaacct attgtaaata
4141 caagttctat ttaagtgtac taattaacat ataatatatg ttttaaatat agaattttta
4201 atgtttttaa atatattttc aaagtacata aaaaaaaaaa aaaaaaa
The WPRE seq in the AAV vectors:
[SEQ ID NO: 4]
ATAATCAACCTCTGGATTACAAAATTTGTGAAAGATTGACTGGTATTCTTAACTATGTT
GCTCCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGAGTT
GTGGCCCGTTGTCAGGCAACGTGGCGTGGTGTGCACTGTGTTTGCTGACGCAACCCCCA
CTGGTTGGGGCATTGCCACCACCTGTCAGCTCCTTTCCGGGACTTTCGCTTTCCCCCTCC
CTATTGCCACGGCGGAACTCATCGCCGCCTGCCTTGCCCGCTGCTGGACAGGGGCTCGG
CTGTTGGGCACTGACAATTCCGTGGTGTTGTCGGGGAAATCATCGTCCTTTCCTTGGCTG
CTCGCCTGTGTTGCCACCTGGATTCTGCGCGGGACGTCCTTCTGCTACGTCCCTTCGGCC
CTCAATCCAGCGGACCTTCCTTCCCGCGGCCTGCTGCCGGCTCTGCGGCCTCTTCCGCGT
CTTCGCCTTCGCCCTCAGACGAGTCGGATCTCCCTTTGGGCCGCCTCCCCGCATC
WPRE 74:
[SEQ ID NO: 5]
CTATGTGGACGCTGCTTTA
WPRE155:
[SEQ ID NO: 6]
TCCTGGTTTGTCTCTTTAT
LOCUS NM_170735; 4755 bp; mRNA; linear; PRI 15-FEB-2009
DEFINITION Homo sapiens brain-derived neurotrophic factor
(BDNF), transcript; variant 1;
mRNA.ACCESSION NM_170735; VERSION NM_170735.5 GI: 219842286
Transcript Variant: This variant (1), also known as IX,
represents the longest transcript. Variants 1,
2, 4, 5, and 7-16 encode the same isoform (a).
[SEQ ID NO: 7]
ORIGIN
      1 cacacacaca cacacacaca gagagaacat ctctagtaaa aagaaaagtt gagctttctt
    61 agctagatgt gtgtattagc cagaaaaagc caaggagtga agggttttag agaactggag
  121 gagataaagt ggagtctgca tatgggaggc atttgaaatg gacttaaatg tctttttaat
  181 gctgactttt tcagttttct ccttaccaga cacattgttt tcatgacatt agccccaggc
  241 atagacacat cattaaaatg aacatgtcaa aaaatgattt ctgtttagaa ataagcaaaa
  301 cattttcagt tgtgaccacc caggtgtaga ataaagaaca gtggaattgg gagccctgag
  361 ttctaacata aactttcttc atgacataag gcaagtcttc tatggccttt ggtttcctta
  421 cctgtaaaac aggatggctc aatgaaatta tctttcttct ttgctataat agagtatctc
  481 tgtgggaaga ggaaaaaaaa agtcaattta aaggctcctt atagttcccc aactgctgtt
  541 ttattgtgct attcatgcct agacatcaca tagctagaaa ggcccatcag acccctcagg
  601 ccactgctgt tcctgtcaca cattcctgca aaggaccatg ttgctaactt gaaaaaaatt
  661 actattaatt acacttgcag ttgttgctta gtaacattta tgattttgtg tttctcgtga
  721 cagcatgagc agagatcatt aaaaattaaa cttacaaagc tgctaaagtg ggaagaagga
  781 gaacttgaag ccacaatttt tgcacttgct tagaagccat ctaatctcag gttatatgct
  841 agatcttggg ggcaaacact gcatgtctct ggtttatatt aaaccacata cagcacacta
  901 ctgacactga tttgtgtctg gtgcagctgg agtttatcac caagacataa aaaaaccttg
  961 accctgcaga atggcctgga attacaatca gatgggccac atggcatccc ggtgaaagaa
1021 agccctaacc agttttctgt cttgtttctg ctttctccct acagttccac caggtgagaa
1081 gagtgatgac catccttttc cttactatgg ttatttcata ctttggttgc atgaaggctg
1141 cccccatgaa agaagcaaac atccgaggac aaggtggctt ggcctaccca ggtgtgcgga
1201 cccatgggac tctggagagc gtgaatgggc ccaaggcagg ttcaagaggc ttgacatcat
1261 tggctgacac tttcgaacac gtgatagaag agctgttgga tgaggaccag aaagttcggc
1321 ccaatgaaga aaacaataag gacgcagact tgtacacgtc cagggtgatg ctcagtagtc
1381 aagtgccttt ggagcctcct cttctctttc tgctggagga atacaaaaat tacctagatg
1441 ctgcaaacat gtccatgagg gtccggcgcc actctgaccc tgcccgccga ggggagctga
1501 gcgtgtgtga cagtattagt gagtgggtaa cggcggcaga caaaaagact gcagtggaca
1561 tgtcgggcgg gacggtcaca gtccttgaaa aggtccctgt atcaaaaggc caactgaagc
1621 aatacttcta cgagaccaag tgcaatccca tgggttacac aaaagaaggc tgcaggggca
1681 tagacaaaag gcattggaac tcccagtgcc gaactaccca gtcgtacgtg cgggccctta
1741 ccatggatag caaaaagaga attggctggc gattcataag gatagacact tcttgtgtat
1801 gtacattgac cattaaaagg ggaagatagt ggatttatgt tgtatagatt agattatatt
1861 gagacaaaaa ttatctattt gtatatatac ataacagggt aaattattca gttaagaaaa
1921 aaataatttt atgaactgca tgtataaatg aagtttatac agtacagtgg ttctacaatc
1981 tatttattgg acatgtccat gaccagaagg gaaacagtca tttgcgcaca acttaaaaag
2041 tctgcattac attccttgat aatgttgtgg tttgttgccg ttgccaagaa ctgaaaacat
2101 aaaaagttaa aaaaaataat aaattgcatg ctgctttaat tgtgaattga taataaactg
2161 tcctctttca gaaaacagaa aaaaacacac acacacacaa caaaaatttg aaccaaaaca
2221 ttccgtttac attttagaca gtaagtatct tcgttcttgt tagtactata tctgttttac
2281 tgcttttaac ttctgatagc gttggaatta aaacaatgtc aaggtgctgt tgtcattgct
2341 ttactggctt aggggatggg ggatgggggg tatatttttg tttgttttgt gttttttttt
2401 cgtttgtttg ttttgttttt tagttcccac agggagtaga gatggggaaa gaattcctac
2461 aatatatatt ctggctgata aaagatacat ttgtatgttg tgaagatgtt tgcaatatcg
2521 atcagatgac tagaaagtga ataaaaatta aggcaactga acaaaaaaat gctcacactc
2581 cacatcccgt gatgcacctc ccaggccccg ctcattcttt gggcgttggt cagagtaagc
2641 tgcttttgac ggaaggacct atgtttgctc agaacacatt ctttcccccc ctccccctct
2701 ggtctcctct ttgttttgtt ttaaggaaga aaaatcagtt gcgcgttctg aaatatttta
2761 ccactgctgt gaacaagtga acacattgtg tcacatcatg acactcgtat aagcatggag
2821 aacagtgatt tttttttaga acagaaaaca acaaaaaata accccaaaat gaagattatt
2881 ttttatgagg agtgaacatt tgggtaaatc atggctaagc ttaaaaaaaa ctcatggtga
2941 ggcttaacaa tgtcttgtaa gcaaaaggta gagccctgta tcaacccaga aacacctaga
3001 tcagaacagg aatccacatt gccagtgaca tgagactgaa cagccaaatg gaggctatgt
3061 ggagttggca ttgcatttac cggcagtgcg ggaggaattt ctgagtggcc atcccaaggt
3121 ctaggtggag gtggggcatg gtatttgaga cattccaaaa cgaaggcctc tgaaggaccc
3181 ttcagaggtg gctctggaat gacatgtgtc aagctgcttg gacctcgtgc tttaagtgcc
3241 tacattatct aactgtgctc aagaggttct cgactggagg accacactca agccgactta
3301 tgcccaccat cccacctctg gataattttg cataaaattg gattagcctg gagcaggttg
3361 ggagccaaat gtggcatttg tgatcatgag attgatgcaa tgagatagaa gatgtttgct
3421 acctgaacac ttattgcttt gaaactagac ttgaggaaac cagggtttat cttttgagaa
3481 cttttggtaa gggaaaaggg aacaggaaaa gaaaccccaa actcaggccg aatgatcaag
3541 gggacccata ggaaatcttg tccagagaca agacttcggg aaggtgtctg gacattcaga
3601 acaccaagac ttgaaggtgc cttgctcaat ggaagaggcc aggacagagc tgacaaaatt
3661 ttgctcccca gtgaaggcca cagcaacctt ctgcccatcc tgtctgttca tggagagggt
3721 ccctgcctca cctctgccat tttgggttag gagaagtcaa gttgggagcc tgaaatagtg
3781 gttcttggaa aaatggatcc ccagtgaaaa ctagagctct aagcccattc agcccatttc
3841 acacctgaaa atgttagtga tcaccacttg gaccagcatc cttaagtatc agaaagcccc
3901 aagcaattgc tgcatcttag tagggtgagg gataagcaaa agaggatgtt caccataacc
3961 caggaatgaa gataccatca gcaaagaatt tcaatttgtt cagtctttca tttagagcta
4021 gtctttcaca gtaccatctg aatacctctt tgaaagaagg aagactttac gtagtgtaga
4081 tttgttttgt gttgtttgaa aatattatct ttgtaattat ttttaatatg taaggaatgc
4141 ttggaatatc tgctatatgt caactttatg cagcttcctt ttgagggaca aatttaaaac
4201 aaacaacccc ccatcacaaa cttaaaggat tgcaagggcc agatctgtta agtggtttca
4261 taggagacac atccagcaat tgtgtggtca gtggctcttt tacccaataa gatacatcac
4321 agtcacatgc ttgatggttt atgttgacct aagatttatt ttgttaaaat ctctctctgt
4381 tgtgttcgtt cttgttctgt tttgttttgt tttttaaagt cttgctgtgg tctctttgtg
4441 gcagaagtgt ttcatgcatg gcagcaggcc tgttgctttt ttatggcgat tcccattgaa
4501 aatgtaagta aatgtctgtg gccttgttct ctctatggta aagatattat tcaccatgta
4561 aaacaaaaaa caatatttat tgtattttag tatatttata taattatgtt attgaaaaaa
4621 attggcatta aaacttaacc gcatcagaac ctattgtaaa tacaagttct atttaagtgt
4681 actaattaac atataatata tgttttaaat atagaatttt taatgttttt aaatatattt
4741 tcaaagtaca taaaa
```

1. A composition for mediation of enrichment-induced tumor resistance comprising: an effective amount of a functional fragment of brain derived neurotrophic factor (BDNF).

2. The composition of claim 1, comprising microRNA BDNF (miR-Bdnf) [SEQ ID NO:1].

3. The composition of claim 1, useful as a mediator linking the environment to metabolic and immune functions and tumor growth.

4. A method for treating a subject with a tumor associated disease, comprising:
administering an expression vector to a target cell in the subject,
wherein the expression vector includes at least a microRNA BDNF (miR-Bdnf) [SEQ ID NO:1], or a derivative or functional fragment thereof, and
wherein administering results in expression of BDNF, or a derivative or functional fragment thereof, in the target cell and the expression reduces the symptoms of the disease, thereby treating the subject with the disease.

5. The method of claim 4, wherein the expression vector is a viral or a non-viral expression vector.

6. The method of claim 4, wherein the viral expression vector is an adeno-associated virus (AAV) vector, a lentivirus vector, an adenovirus vector, or a herpes simplex virus (HSV) vector.

7. The method of claim 4, wherein the disease is cancer.

8. The method of claim 4, wherein the disease is colon cancer.

9. The method of claim 4, wherein the disease is melanoma.

10. The method of claim 4, wherein the disease is diabetes.

11. The method of claim 4, wherein the disease is obesity.

12. The method of claim 4, wherein the administering is by stereotaxic microinjection.

13. The method of claim 4, wherein the administering is by stereotaxic microinjection to a medial temporal lobe or temporal cortex of the central nervous system of the subject.

14. The method of claim 4, wherein the administering is by stereotaxic microinjection to the medial temporal lobe is localized to the hippocampus and/or amygdale of the subject.

15. A method for delivering a nucleic acid sequence to a target cell, wherein the nucleic acid sequence is expressible in the target cell, the method comprising: administering a BDNF-associated vector to the target cell.

16. The method of claim 15, wherein the vector transduces the target cell; and wherein the vector is free of both wildtype and helper virus.

17. The method of claim 15, wherein the BDNF-associated vector comprises microRNA BDNF (miR-Bdnf) [SEQ ID NO:1].

18. (canceled)

19. (canceled)

20. (canceled)

21. A method of altering expression of brain derived neurotrophic factor (BDNF) in of a subject comprising:
identifying a target site in the subject that requires modification;
delivering a vector comprising a nucleotide sequence encoding a an effective amount of a functional fragment of brain derived neurotrophic factor (BDNF) to the target site sufficient to allow expression of BDNF in the target site.

22. The method of claim 21, wherein the vector is a viral vector.

23. The method of claim 21, wherein the vector is delivered using stereotaxic delivery.

24. The method of claim 21, wherein the target site is the central nervous system of the subject.

25. The method of claim 21, wherein the target site is a region of the brain selected from the group consisting of basal ganglia, subthalmic nucleus (STN), pedunculopontine nucleus (PPN), substantia nigra (SN), thalmus, hippocampus, cortex, and combinations thereof.

26. (canceled)

27. (canceled)

28. (canceled)

29. (canceled)

30. (canceled)

31. (canceled)

32. A method of inducing an immune response in a subject comprising administering to the subject a vaccine comprising an effective amount of a functional fragment of brain derived neurotrophic factor (BDNF).

33. (canceled)

34. (canceled)

35. The method of claim 32, wherein the disorder is one or more of: obesity, metabolic syndrome, cancer, and other conditions involving degeneration or dysfunction of cells expressing BDNF.

36. The method of claim 32, wherein the subject is a human subject.

37. (canceled)

38. (canceled)

39. (canceled)

40. (canceled)

41. A method for treating obesity comprising:
identifying a target site in a brain for modification of a subject in need thereof;
transfecting at least one cell at the target site with a vector expressing a therapeutic protein comprised of brain derived neurotrophic factor (BDNF); and,
expressing the therapeutic protein in an amount effective for modulating metabolism in the subject.

42. The method of claim 41, wherein the target site of the brain is at least a hypothalamus.

43. A pharmaceutical composition for treating a metabolic disorder comprising:
an effective amount of an adeno-associated viral vector encoding at least a portion of a gene to increase or decrease expression of a therapeutic protein comprised of brain derived neurotrophic factor (BDNF) in a desired region of a brain; and,
a pharmaceutically acceptable carrier to treat the metabolic disorder.

44. (canceled)

45. A method of treating one or more of obesity and related metabolic syndromes, comprising: administering an AAV-BDNF vectors comprised of:
i) an autoregulatory negative feedback system using RNAi coupled to transgene-induced physiological changes, and
ii) a definitive knockout via delivery of a second, rescue vector based on Cre-loxP systems.

46. (canceled)

47. A method for reducing colon cancer in a subject, comprising providing an environmental enrichment sufficient to reduce colon cancer growth comprising: upregulating hypothalamic BDNF after the cancer has been established in the subject.

48. A method for treating obesity and cancers associated with obesity in a subject, comprising the hypothalamic gene delivery of an AAV-BDNF vector to the subject.

49. The method of claim 48, comprising providing the AAV-BDNF therapeutic vector by combining an autoregulatory strategy with a rescue strategy.

50. The method of claim 49, wherein the autoregulatory strategy comprises RNAi mimicking physiological negative feedback.

51. The method of claim 49, wherein the rescue strategy comprises a knockout via Cre-loxP.

52. (canceled)

53. (canceled)

54. The method of claim 51, including an autoregulatory negative feedback system using RNAi coupled to transgene-induced physiological changes and a definitive knockout via delivery of a second, rescue vector based on Cre-loxP systems.

55. A vector expressing microRNA BDNF (miR-Bdnf) [SEQ ID NO:1].
